(19) United States
(12) Patent Application Publication
(10) Pub. No.: US 2017/0080128 A1
(43) Pub. Date: Mar. 23, 2017

(54) NOVEL ENDOTRACHEAL TUBE FOR THE REDUCTION OF INTUBATION-RELATED COMPLICATION IN NEONATES AND BABIES

(71) Applicant: Brigham Young University, Provo, UT (US)

(72) Inventors: Carl Genberg, Las Vegas (UT); Paul B. Savage, Mapleton, UT (US); Ronald L. Bracken, Monroe, GA (US)

(21) Appl. No.: 15/270,876

(22) Filed: Sep. 20, 2016

Related U.S. Application Data
(60) Provisional application No. 62/221,613, filed on Sep. 21, 2015.

(51) Int. Cl. A61L 29/16; A61L 29/06; A61F 11/00; A61M 25/00; A61M 16/04; A61J 15/00; A61L 29/14; A61L 29/08

(52) U.S. Cl. CPC A61L 29/16; A61L 29/145; A61L 29/06; A61L 29/14; A61L 29/08; A61M 25/00; A61M 16/0465; A61J 15/00; A61F 11/002; A61L 2400/10; A61L 2300/404; A61L 2300/222; A61L 2300/41; A61L 2300/606; A61M 2205/0205; A61M 2205/0238; A61M 2207/00

(57) ABSTRACT

This disclosure relates to medical devices incorporating one or more cationic steroidal antimicrobials (CSAs). The CSAs are incorporated into the medical devices to provide effective antimicrobial, anti-inflammatory, and/or tissue-healing properties. A medical device includes a component formed from a polymeric material. One or more CSA compounds are mixed with the polymeric material so that the one or more CSA compounds are incorporated into the structure of the medical device as formed from the polymeric material. A medical device can additionally or alternatively include a lubricious coating containing one or more CSA compounds.

### CROSS-REFERENCE TO RELATED APPLICATIONS

This application claims the benefit of U.S. Provisional Patent Application Ser. No. 62/221,613, filed on Sep. 21, 2015, the disclosure of which is incorporated herein in its entirety.

### BACKGROUND

1. Field of Disclosure

The disclosure relates generally to medical devices, including in particular implantable medical devices, which incorporate one or more cationic steroidal antimicrobial (CSA) compounds to provide one or more of anti-microbial activity, anti-inflammatory activity, reduced pain, and increased rate of tissue healing.

2. Related Technology

Medical devices include instruments used on a subject's body for diagnostic or therapeutic purposes. In use, many medical devices are implanted into the subject, and may be intended either as a permanent or temporary implant. However, even when strict sterilization procedures are followed, such medical implants can be subject to microbial contamination (e.g., biofilm formation). When biofouling of the implant occurs, the implant must be removed from the subject. After fouling, the medical device is typically unfit for any further use, and must be discarded. Usually, the fouled implant must be replaced with a new implant, adding to medical care costs both by requiring the purchase of the new implant and for associated costs of inserting the implant.

Further, fouling of an implanted medical device is often associated with detrimental health effects. In many circumstances, an implant serves as a site for microbial contamination and biofilm formation, which leads to recurrent and difficult to manage infections. These infections can occur at tissue sites near the implant, or can even occur at other remote locations in the subject's body. A microbial infection associated with a fouled implant can cause serious health problems for the patient, and can even lead to very serious and deadly conditions, such as sepsis. Even when treatable, these implant-associated infections require additional medical care, with its concomitant costs, prolonged healing times, and patient discomfort.

In addition, the implantation of a medical device can trigger an inflammatory response from the subject, even in the absence of any corresponding implant-related infection. This can occur as a result of the subject's reaction to the introduction of an unknown and foreign object. In many instances, even if the implant itself is bio-inert, the manner in which it is deployed requires the implant to be positioned against or within surrounding tissues, which can aggravate the surrounding tissues and lead to inflammation and pain.

One example of medical devices subject to such issues are endotracheal tubes (ETTs). ETTs are catheter devices inserted through the mouth or nose of a patient to establish and maintain the patient's airway for adequate breathing, and are frequently used during periods where the patient is under mechanical ventilation (assisted breathing). Commonly, patients undergoing mechanical ventilation are premature infants. ETTs are particularly prone to microbial colonization and fouling. Use of ETTs is also associated with a host of complications, particularly bronchopulmonary dysplasia (BPD) and subglottic stenosis.

BPD is a neonatal form of chronic lung disease and is associated with an increased risk of pulmonary and neurologic impairment, which in preterm infants can persist into adulthood. Damage caused by BPD can persist for many years, causing premature aging of the lungs, oxygen-dependency, high hospital readmission rates, and high rates of symptomatic airway obstruction.

Subglottic stenosis is the narrowing of the airway below the vocal cords and above the trachea. Bacterial biofilms are particularly suspected as playing a role in the development of subglottic stenosis. Correcting subglottic stenosis requires surgery to expand the lumen of the cricoid area, which increases airflow and decreases obstructive breathing. While less severe cases of subglottic stenosis can be corrected with endoscopic laser surgery, more severe cases require reconstruction with cartilage grafts and stents.

### BRIEF SUMMARY

Disclosed herein are implantable medical devices incorporating one or more cationic steroidal antimicrobial (CSA) compounds to provide the medical device with effective antimicrobial properties and/or anti-inflammatory properties. In some embodiments, implantable medical devices incorporating one or more CSA compounds are additionally or alternatively provided with effective analgesic properties and/or tissue healing properties. In some embodiments, implantable medical devices incorporate one or more CSA compounds capable of exhibiting anti-inflammatory properties independently of any antimicrobial properties.

Non-limiting examples of implantable medical devices which may incorporate one or more CSA compounds, as described herein, include catheters, endotracheal tubes, intravenous feed lines, feeder tubes, drains, prosthesis components (e.g., voice prostheses), peristaltic pumps, tympanostomy tubes, tracheostomy tubes, and the like. Examples of implantable medical devices additionally include medical devices which, in use, are implanted into a subject's tissues, deployed at a puncture or wound site, positioned for feeding or withdrawing material from a body cavity, or are otherwise associated with a subject in such a way that biological compatibility is of concern (e.g., because infection and/or inflammation can result).

In some embodiments, an implantable medical device including CSA compounds provides antimicrobial properties, and thereby provides the benefits of reducing fouling of the implant, reducing infection risk associated with fouling of the implant, reducing infection-related inflammation associated with the implant, reducing patient discomfort associated with an infection, and/or enabling more positive outcomes following a medical treatment involving an implanted medical device.

In some embodiments, an implantable medical device including CSA compounds provides the benefits of reducing inflammation, reducing pain, and/or increasing the rate of tissue healing in the absence of any microbial contamination or infection. Thus, at least some of the medical devices described herein provide, independently, the benefits of anti-microbial functionality, anti-inflammatory functionality, analgesic functionality, and tissue healing functionality.

Without being bound to theory, it is believed that in at least some applications an increased rate of tissue healing is caused by increases in fibroblastic migration and enhanced epithelial growth factors at the tissue site. Subjects have also exhibited a significantly reduced sensitivity to pain. In some embodiments, the therapeutic anti-inflammatory effect is derived from the steroid-like structure of the CSA compounds and/or effects in modulating genes related to inflammation, and the anti-inflammatory effect is independent of any anti-microbial activity. However, anti-inflammatory activity may additionally be exhibited as a result of anti-microbial effects of the CSA compounds.

One or more embodiments are directed to methods of controlling microbial growth on a medical device and/or at an implantation site at which an implantable medical device is implanted, and likewise controlling the spread of microbial growth to other areas of a subject's body (e.g., when an infection becomes septic). For example, one or more embodiments are directed to controlling biofilm formation on an implantable medical device. In some embodiments, a method includes (1) implanting an implantable medical device having one or more incorporated CSA compounds, as described herein, and (2) the implantable medical device killing one or more microbes contacting the implantable medical device. The implantable medical device may be effective in killing a wide variety of microbes (e.g., a wide variety of different bacterial strains).

One or more embodiments are directed to methods of reducing inflammation at an implantation site at which a medical device is implanted. In some embodiments, a method includes (1) implanting a medical device having one or more incorporated CSA compounds, as described herein, and (2) the implantable medical device reducing or preventing inflammation at the treatment site (e.g., as compared to a similar implantable medical device not incorporating CSA compounds).

One or more embodiments are directed to methods of increasing the rate of tissue healing at an implantation site at which a medical device has been implanted. In some embodiments, a method includes (1) implanting a medical device having one or more incorporated CSA compounds, as described herein, and (2) the implantable medical device increasing the rate tissue healing at the implantation site (e.g., as compared to a similar implantable medical device not incorporating CSA compounds).

One or more embodiments are directed to methods of manufacturing an implantable medical device with one or more incorporated CSA compounds. In some embodiments, such a method includes: (1) providing an implantable medical device; and (2) applying a coating to at least a portion of a surface of the medical device to associate the coating with the medical device, the coating being formulated with one or more CSA compounds.

In some embodiments, a method of manufacturing a medical device with one or more incorporated CSA compounds includes: (1) providing a biologically compatible moldable polymeric material; (2) mixing one or more CSA compounds with the moldable polymeric material; and (3) molding the moldable polymeric material into an implantable medical device. In preferred embodiments, the one or more CSA compounds are provided in a salt form, such as a naphthalenedisulfonic acid (NDSA) salt, including 1,5-NDSA salt. In some embodiments, molding the moldable polymeric material includes extruding the material through an extruder. In other embodiments, the medical device is formed using injection molding or other polymer molding or shaping process known in the art. By way of example, the CSA compound can be an NDSA salt of CSA-131 and similar CSA compounds.

As used herein, the term “polymeric material” can refer to an already-formed polymer or to a polymerizable material or mixture that is capable of forming a polymer upon activation, curing, setting, etc. The polymeric material may be any polymer or polymerizable material suitable for medical use as part of an implantable medical device, including thermoplastic or thermoset materials. Preferred embodiments are directed to implantable medical devices formed at least partly of silicone. Other medical device embodiments may include polyethylene, polypropylene, polystyrene, polyester, polycarbonate, polyvinyl chloride, polyacrylate, polysulfone, or combinations thereof.

Additional features and advantages will be set forth in part in the description that follows, and in part will be obvious from the description, or may be learned by practice of the embodiments disclosed herein. It is to be understood that both the foregoing brief summary and the following detailed description are exemplary and explanatory only and are not restrictive of the embodiments disclosed herein or as claimed.

### BRIEF DESCRIPTION OF DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIGS. 1A-1C illustrate example cationic steroidal antimicrobial compounds; and

FIG. 2 compares histological images of tracheal tissue of a pre-term lamb intubated with an ETT not coated with a CSA-containing coating to tracheal tissue of a pre-term lamb intubated with an ETT coated with a CSA-containing coating.

### DETAILED DESCRIPTION

### I. Overview of CSA Compounds

Cationic sterioidal antibiotic (“CSA”) compounds (“CSAs”), which are also known as “ceragenin” compounds (or “ceragenins”), are synthetically produced small molecule chemical compounds that include a sterol backbone having various charged groups (e.g., amine, guanidine, and/or other groups capable of exhibiting cationic properties under biological conditions) attached to the backbone. The backbone can be used to orient the cationic groups on one face, or plane, of the sterol backbone.

CSAs are cationic and amphiphilic, based upon the functional groups attached to the backbone. They are facially amphiphilic with a hydrophobic face and a polycationic face. Without wishing to be bound to any particular theory, it is theorized that the CSA compounds described herein act as anti-microbial agents (e.g., anti-bacterials, anti-fungals, and anti-virals) by binding to the cellular membrane of bacteria and other microbes and inserting into the cell membrane, forming a pore that allows the leakage of ions and cytoplasmic materials that are critical to the microbe's survival, thereby leading to the death of the affected microbe. In addition, the CSA compounds described herein may also act to sensitize bacteria to antibiotics. For example, at concentrations of the CSA compounds below the corresponding minimum bacteriostatic concentration, CSAs have been shown to cause bacteria to become more susceptible to other antibiotics by increasing the permeability of the membrane of the bacteria.

The charged groups are responsible for disrupting the bacterial cellular membrane, and without the charged groups, the CSA compound cannot disrupt the membrane to cause cell death or sensitization. An example of a CSA compound is shown below as Formula I. As will be discussed in greater detail below, the R groups of Formula I can have a variety of different functionalities, thus providing a given ceragenin compound with specific, different properties. In addition, as will be appreciated by those of skill in the art, the sterol backbone can be formed of 5-member and/or 6-member rings, so that p, q, m, and n may independently be 1 (providing a 6-member ring) or 0 (providing a 5-member ring).

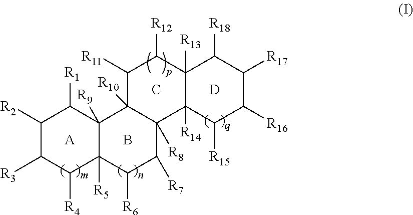

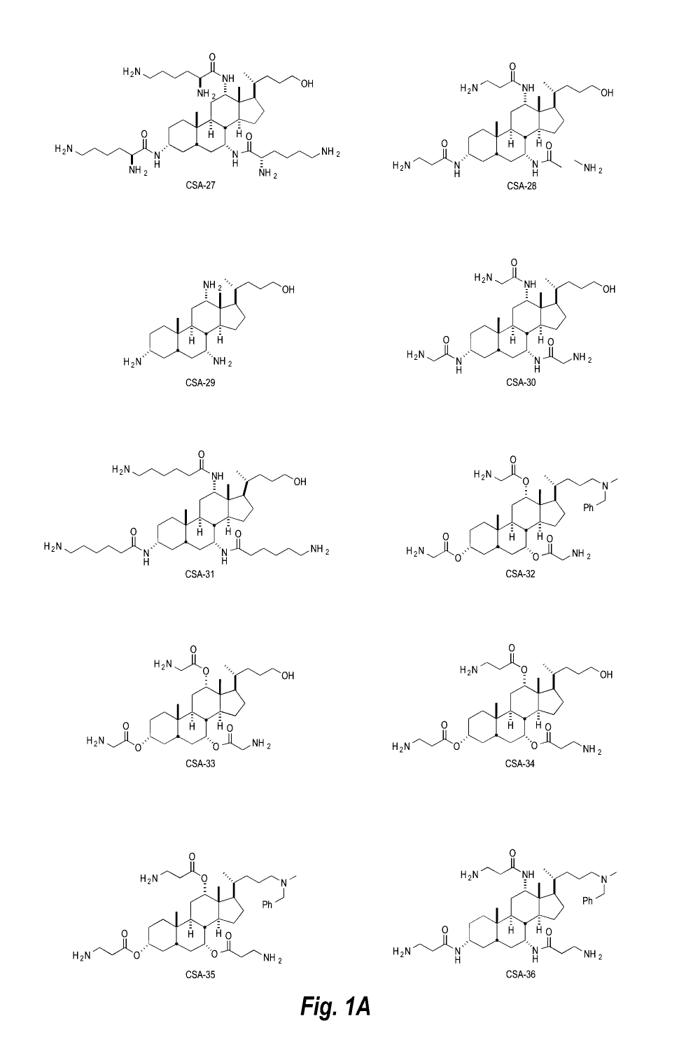

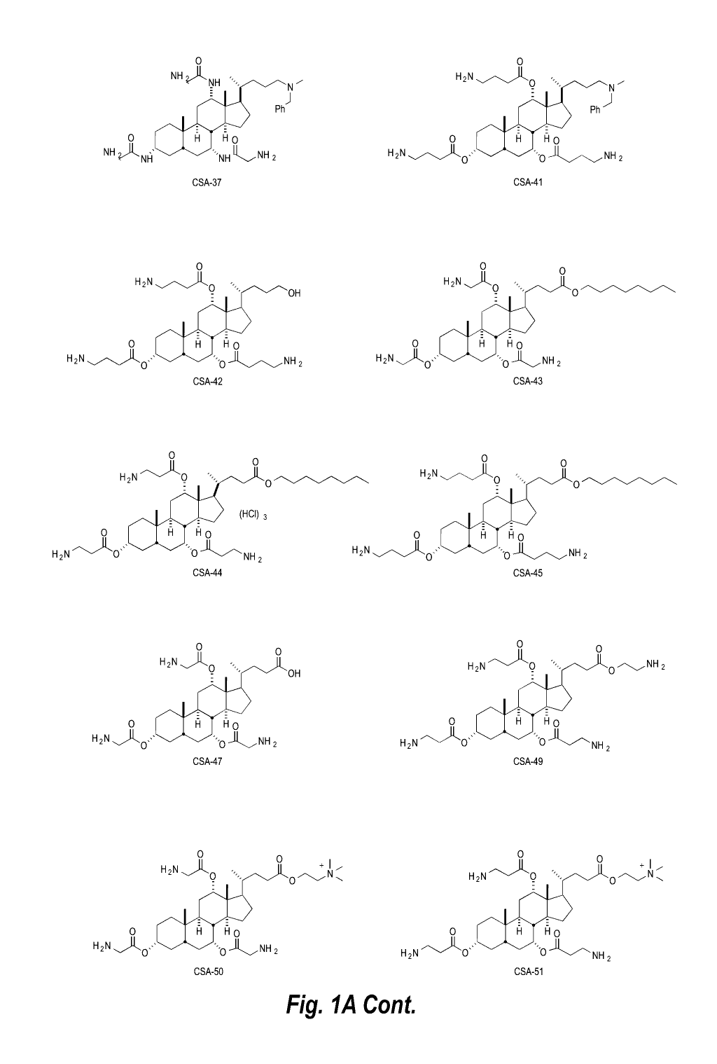

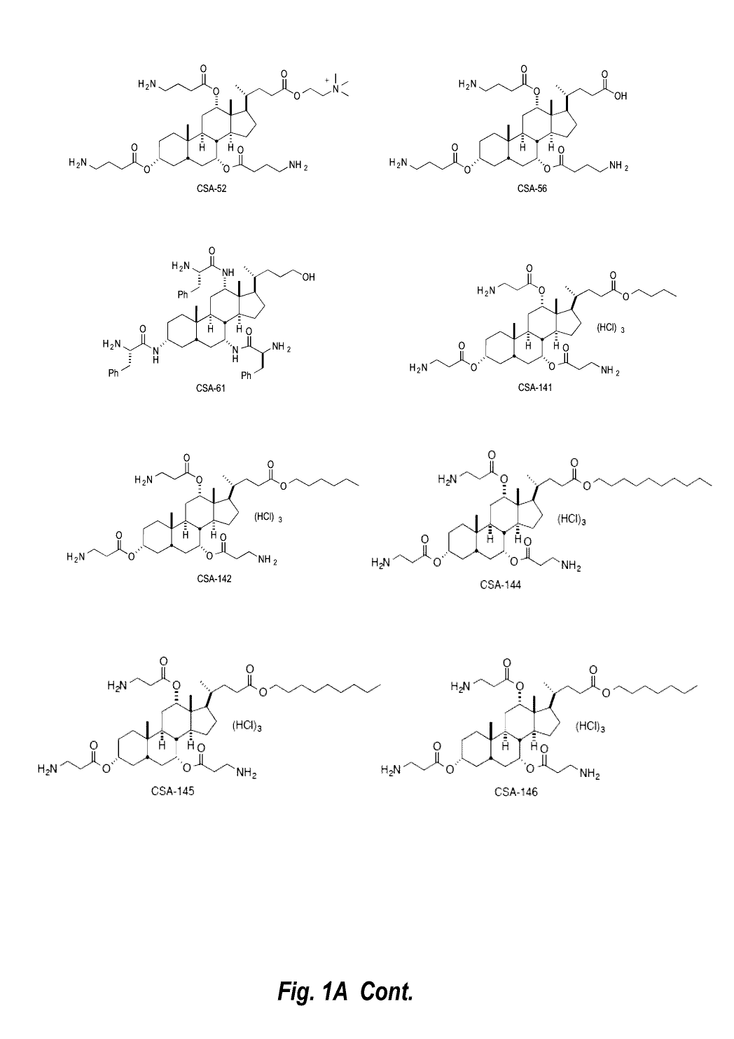

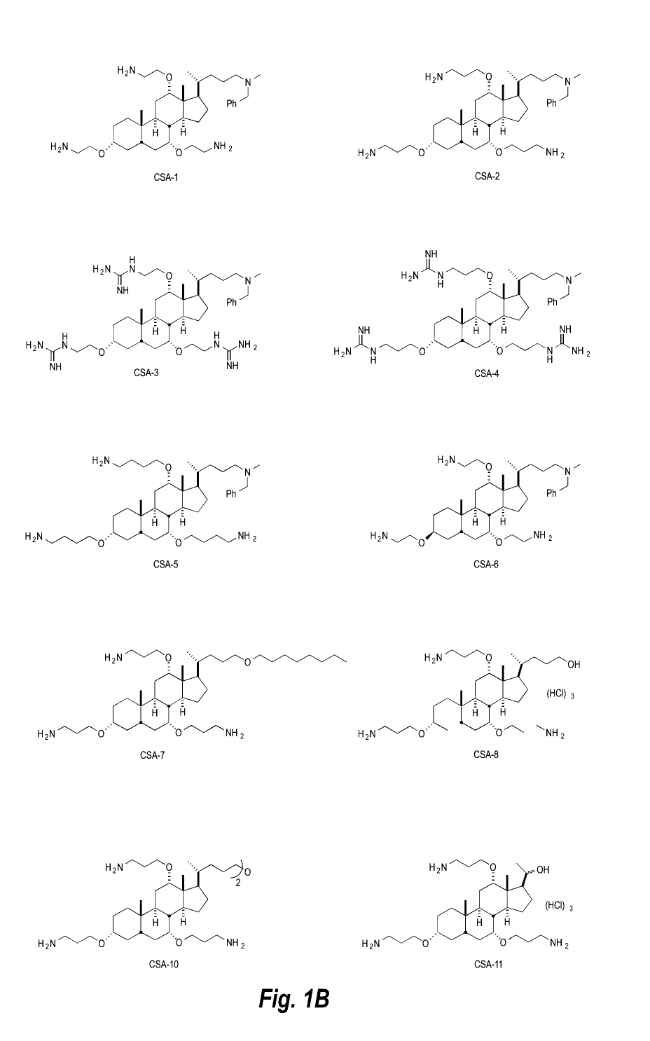

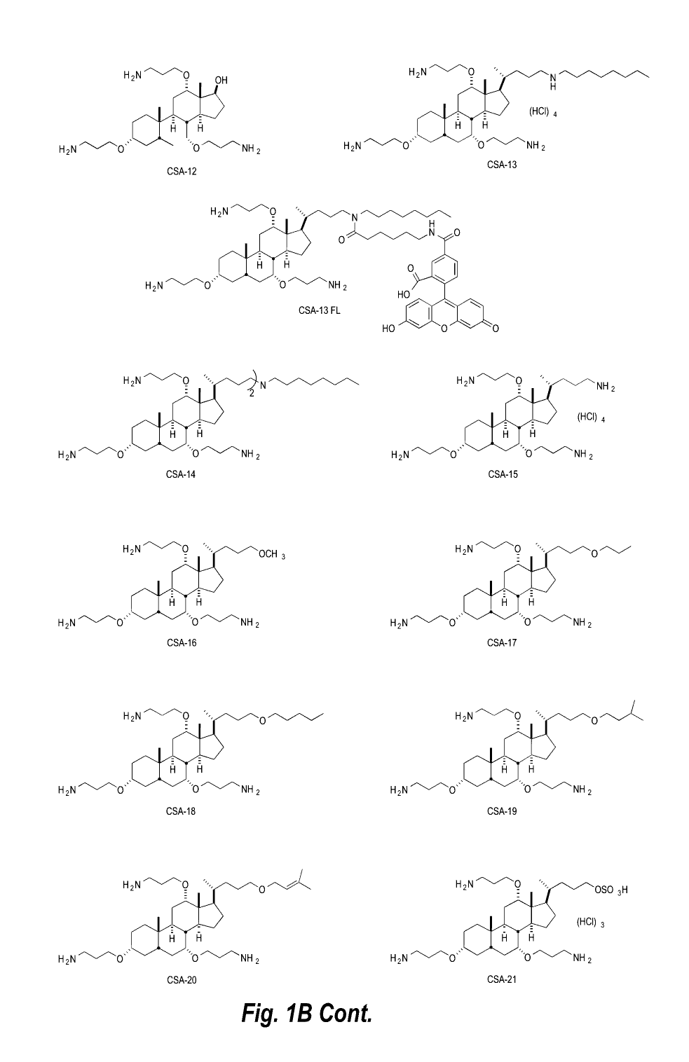

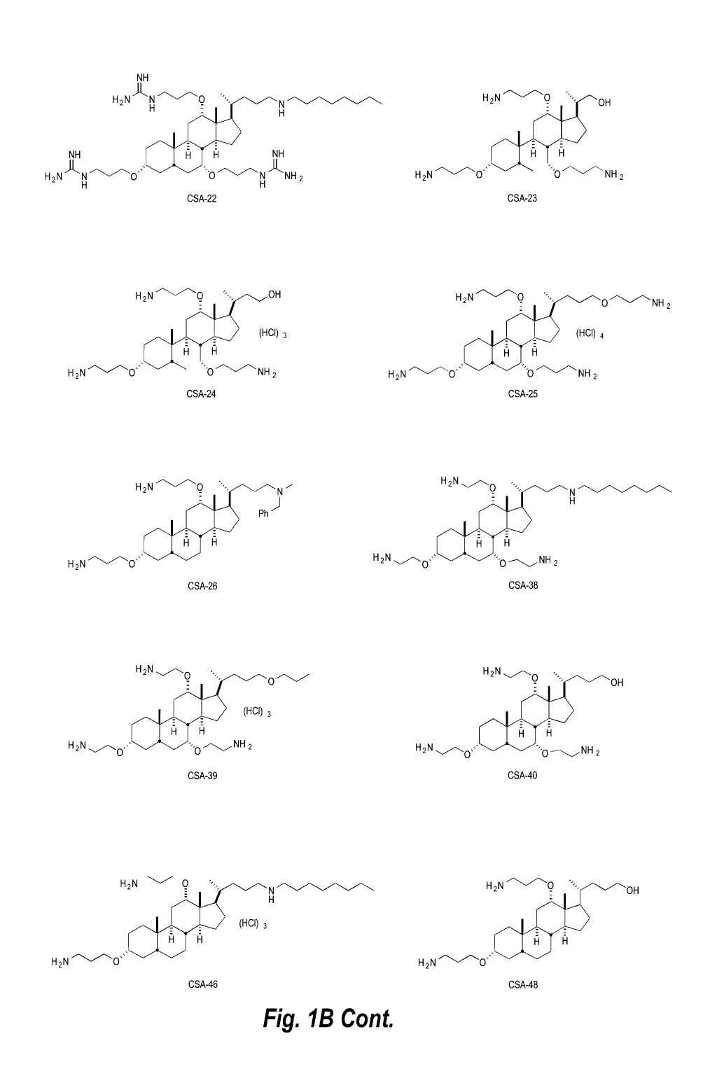

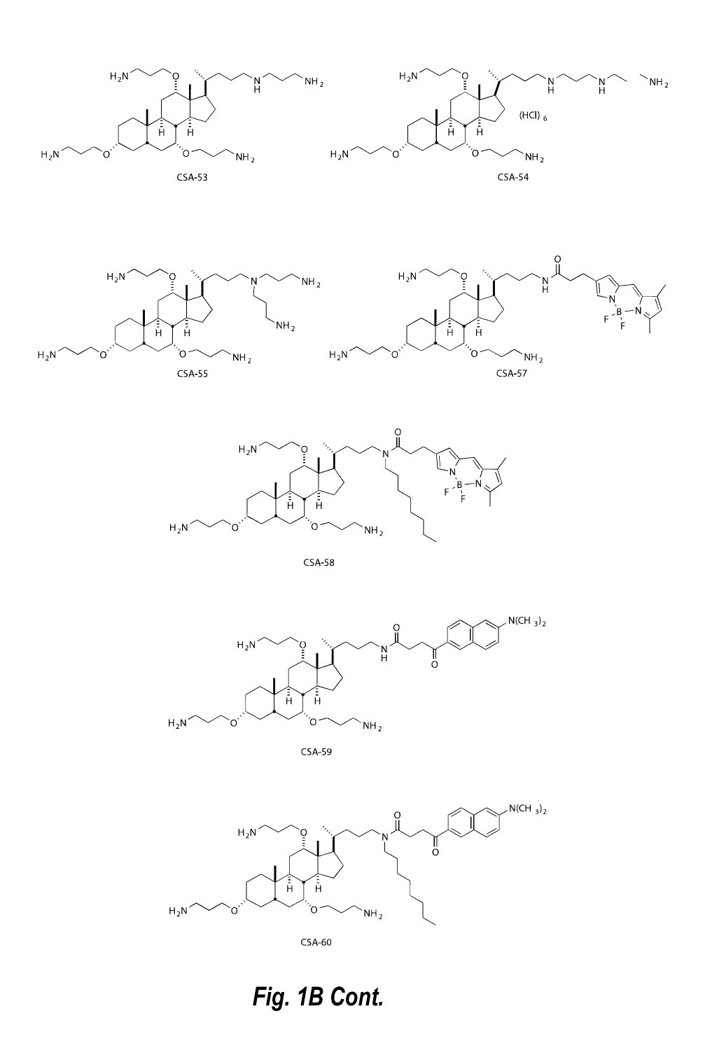

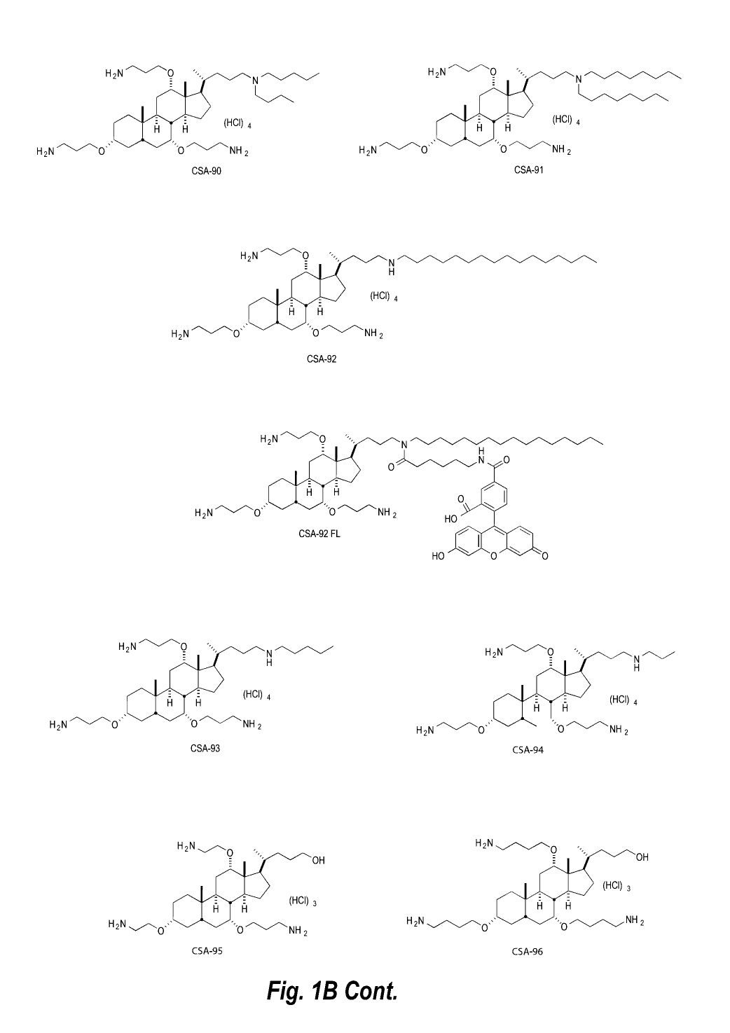

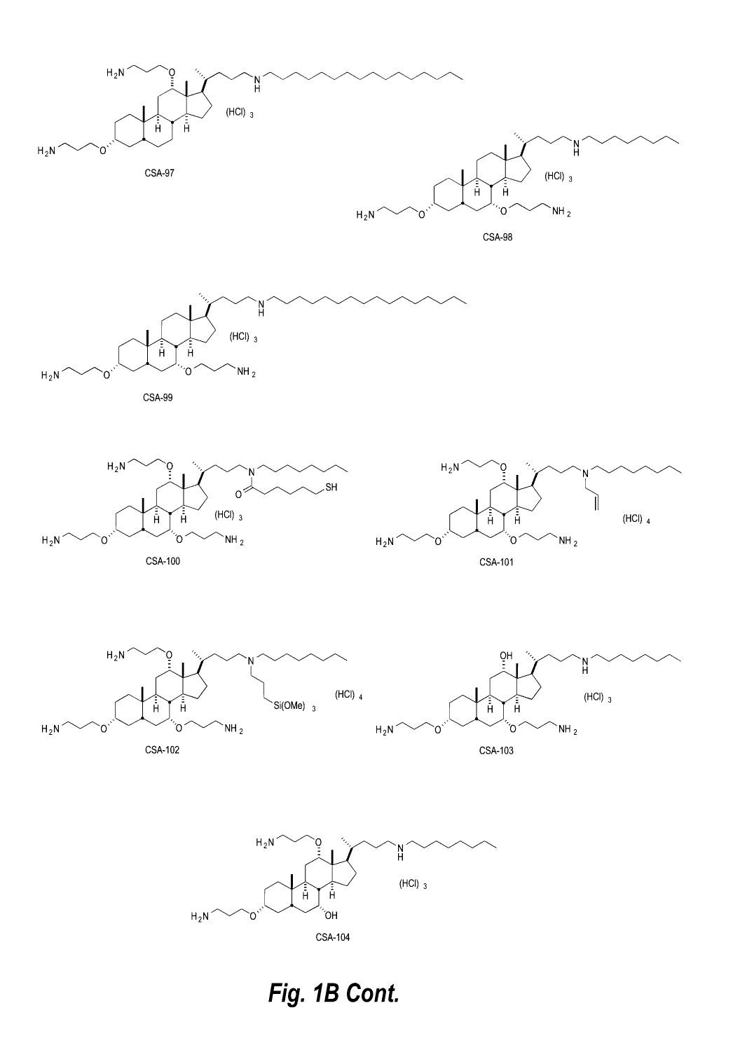

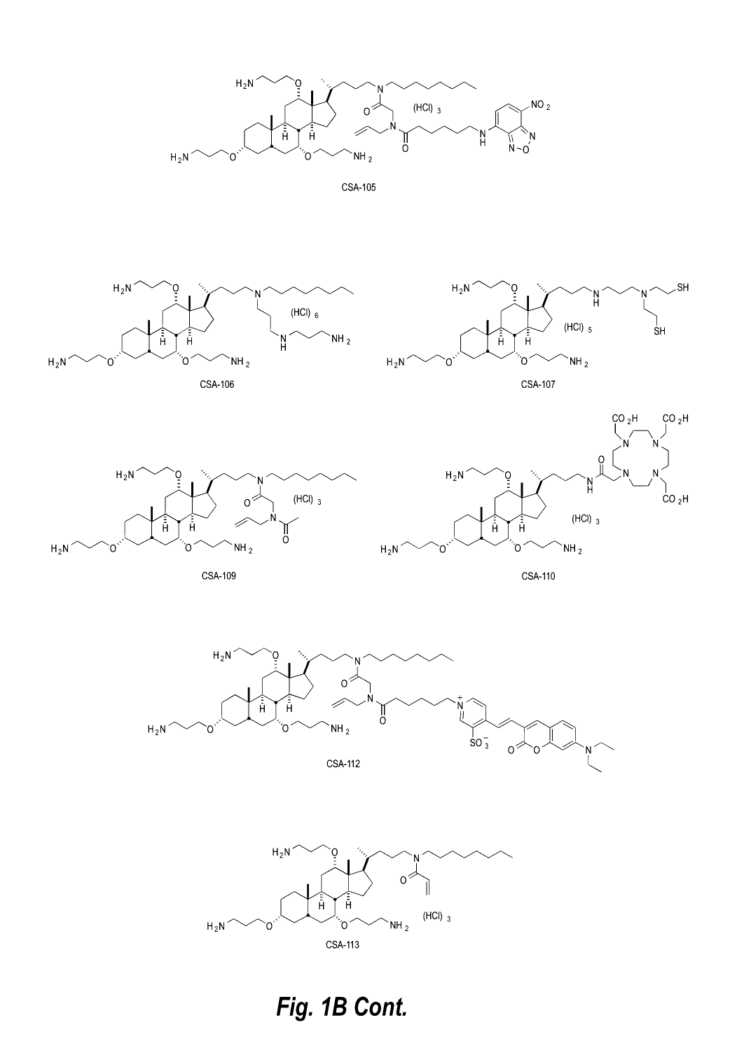

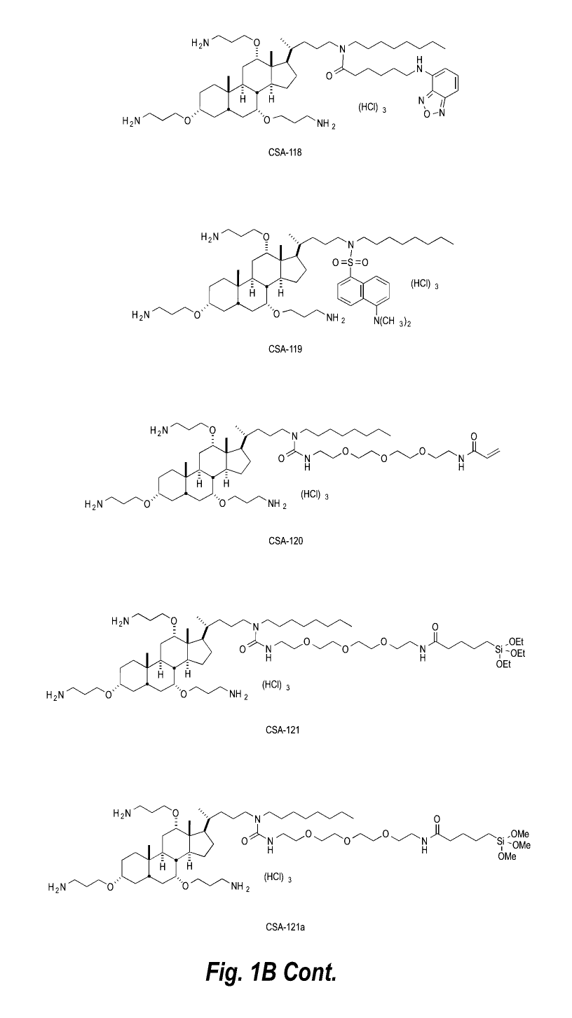

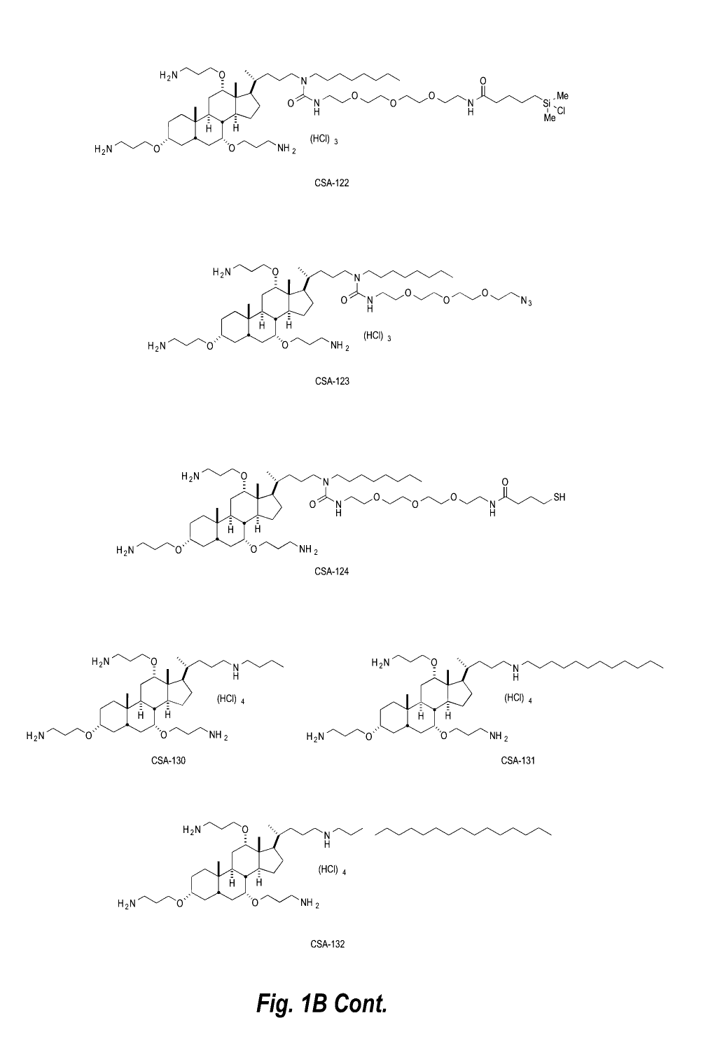

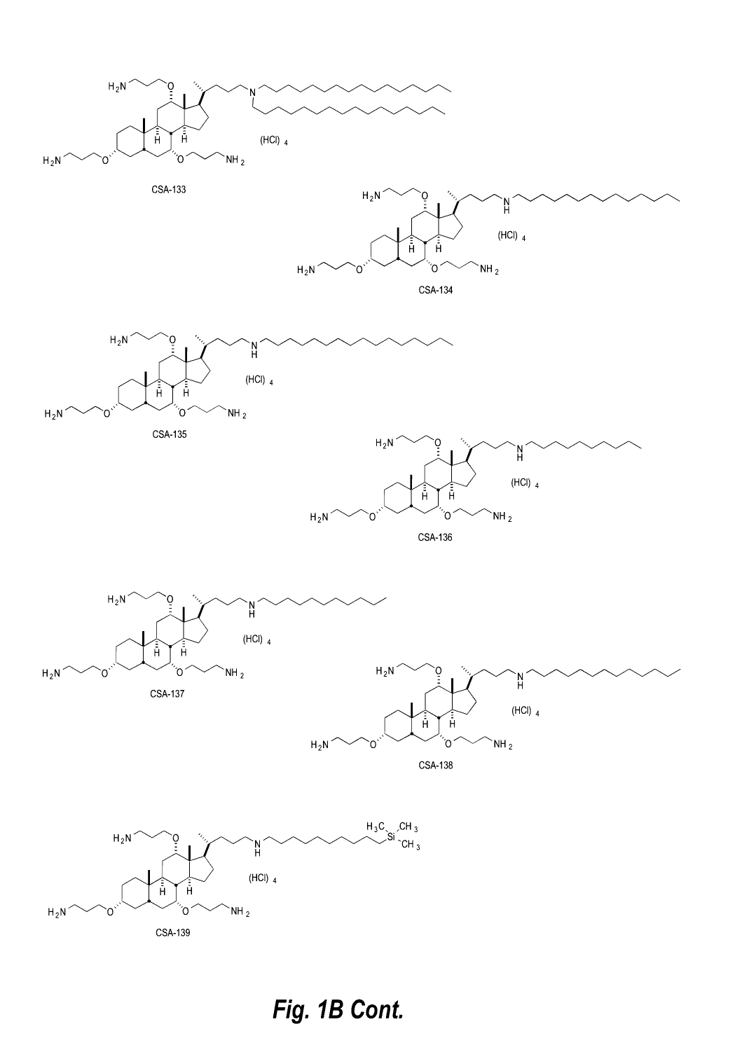

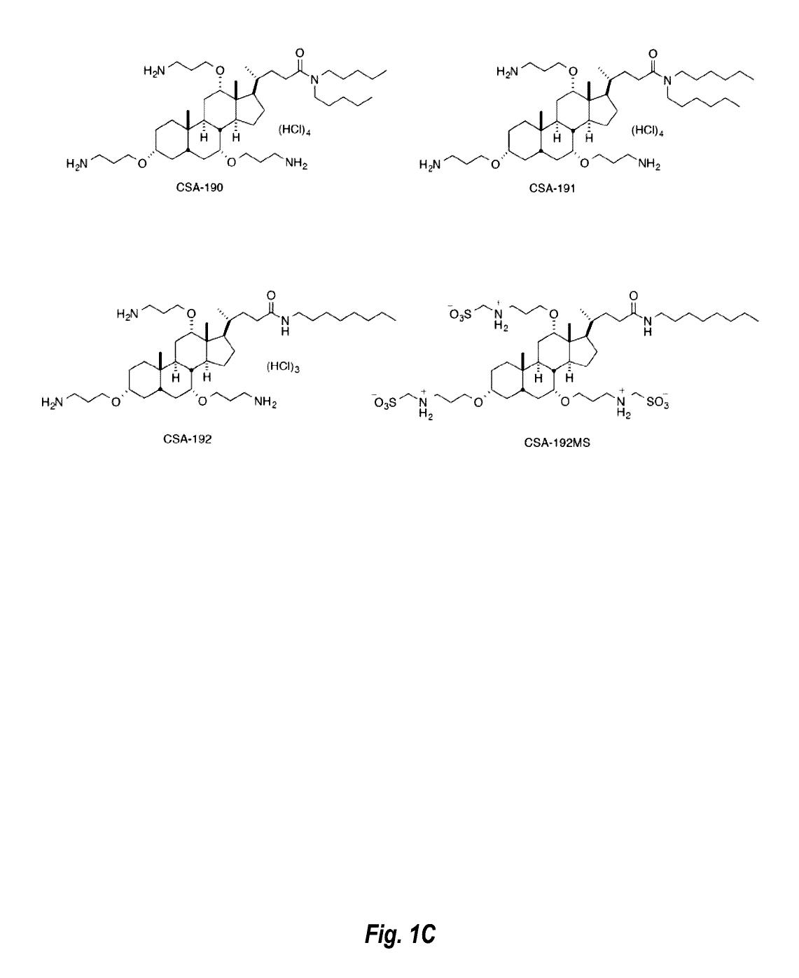

A number of examples of CSA compounds of Formula I that can be incorporated into the medical devices described herein are illustrated in FIGS. 1A-1C.

In addition to effective antimicrobial properties, at least some CSAs have been shown to exhibit effective anti-inflammatory properties. In some instances, effective anti-inflammatory effects of CSAs may correspond to the effective antimicrobial effects of the CSAs, such as when the reduction or elimination of a microbial infection lessens a subject's inflammatory reaction against the infection. At least some CSA formulations have also been shown to provide anti-inflammatory effects independent of any antimicrobial effects. For example, at least some CSA formulations have been shown to be capable of reducing an inflammatory response

Typically, the CSAs of Formula I are of two types: (1) CSAs having cationic groups linked to the sterol backbone with hydrolysable linkages and (2) CSAs having cationic groups linked to the sterol backbone with non-hydrolysable linkages. For example, one type of hydrolysable linkage is an ester linkage, and one type of non-hydrolysable linkage is an ether linkage. CSAs of the first type can be “inactivated” by hydrolysis of the linkages coupling the cationic groups to the sterol backbone, whereas CSAs of the second type are more resistant to degradation and inactivation.

In some applications, it may be desirable for a medical device to maintain antimicrobial and/or anti-inflammatory effects for as long as possible. For example, relatively long term use of medical devices such as catheters, endotracheal tubes, and voice prostheses provides ample opportunity for fouling or introduction of infection and/or inflammation. In many instances, the lifespan of these medical devices is essentially limited to how long they can resist fouling before becoming hazardous to the subject. Accordingly, enhancing the capability to resist microbial colonization and fouling for months, weeks, or even days can decrease medical care costs in addition to decreasing infection and/or inflammation risks.

In other applications, the spreading of eluted CSA compounds beyond the implant site of the medical device may be a concern. Medical device embodiments can be formed using an appropriate mixture of CSAs having hydrolysable and non-hydrolysable linkages to provide desired duration of CSA activity once the CSAs are exposed to biological conditions (e.g., once eluted from the medical device).

A number of examples of compounds of Formula I that may be used in the embodiments described herein are illustrated in FIGS. 1A-1C. Examples of CSAs with non-hydrolysable linkages include, but are not limited to CSA-1, CSA-26, CSA-38, CSA-40, CSA-46, CSA-48, CSA-53, CSA-55, CSA-57, CSA-60, CSA-90, CSA-107, CSA-109, CSA-110, CSA-112, CSA-113, CSA-118, CSA-124, CSA-130, CSA-131, CSA-139, CSA-190, CSA-191 and CSA-192. Suitable examples of CSAs with hydrolysable linkages include, but are not limited to CSA-27, CSA-28, CSA-29, CSA-30, CSA-31, CSA-32, CSA-33, CSA-34, CSA-35, CSA-36, CSA-37, CSA-41, CSA-42, CSA-43, CSA-44, CSA-45, CSA-47, CSA-49, CSA-50, CSA-51, CSA-52, CSA-56, CSA-61, CSA-141, CSA-142, CSA-144, CSA-145 and CSA-146. In a preferred embodiment, at least a portion of the CSA compounds incorporated into the medical device are CSA-131. In other embodiments, the CSA compounds may include CSA-192. Additional details relating to CSA compounds are described below.

In some embodiments, the one or more CSA compounds may have a structure as shown in Formula I. In Formula I, at least two of R₃, R₇, or R₁₂ may independently include a cationic moiety attached to the Formula I structure via a hydrolysable (e.g., an ester) or non-hydrolizable (e.g., an ether) linkage. Optionally, a tail moiety may be attached to Formula I at R₁₈. The tail moiety may be charged, uncharged, polar, non-polar, hydrophobic, or amphipathic, for example, and can thereby be selected to adjust the properties of the CSA and/or to provide desired characteristics.

The anti-microbial activity of the CSA compounds can be affected by the orientation of the substituent groups attached to the backbone structure. In one embodiment, the substituent groups attached to the backbone structure are oriented on a single face of the CSA compound. Accordingly, each of R₃, R₇, and R₁₂ may be positioned on a single face of Formula I. In addition, R₁₈ may also be positioned on the same single face of Formula I.

In some embodiments, the one or more CSA compounds are included by weight in a coating or a polymeric mixture at about 0.1%, 0.5%, 1%, 3%, 5%, 10%, 15%, 20%, 25%, or 30% or are included by weight within a range defined by any two of the foregoing values.

Another advantageous characteristic associated with one or more of the CSA compositions described herein is their effectiveness in killing biofilm type bacteria, in addition to planktonic bacteria. Many other anti-microbial agents suitable for application to a live subject, including nearly all antibiotics, have limited effectiveness in killing bacteria present in a biofilm form. This is believed to be due to the fact that most of such antibiotics attack enzymes associated with growth of bacteria. Biofilm bacteria are believed to be in something of a sessile state so that the targeted growth enzymes are not being produced. This results in the biofilm bacteria surviving an antibiotic treatment, meaning they are capable of continuing to pose a pathogenic threat even after treatment with such antibiotics. The CSA compounds operate through a different mechanism, which is effective against both planktonic and biofilm type bacteria.

In preferred embodiments, the CSA compounds used herein are provided in salt form. It has been found that certain salt forms of CSAs exhibit beneficial properties such as improved solubility, crystallinity, flow, and storage stability. Some embodiments are directed to a sulfuric acid addition salt or sulfonic acid addition salt of a CSA. In some embodiments, the sulfonic acid addition salt is a disulfonic acid addition salt. In some embodiments, the sulfonic acid addition salt is a 1,5-naphthalenedisulfonic acid (NDSA) addition salt, such as an NDSA salt of CSA-131 and/or an NDSA salt of CSA-192. In some embodiments, the acid addition salt is a mono-addition salt. In other embodiments, the acid addition salt is a di-addition salt. In other embodiments, the acid addition salt is a tetra-addition salt.

### II. Medical Devices Incorporating CSA Compounds

As used herein, an “implantable medical device” refers to a medical device that may be implanted into a subject's tissues, deployed at a puncture or wound site, positioned for feeding or withdrawing material from a body cavity, or may otherwise be associated with a subject in such a way that biological compatibility is of concern (e.g., because infection and/or inflammation can result). It will be understood that such an implantable medical device need not be fully implanted within a subject's body. For example, portions of the implant may extend beyond the subject and/or may be associated with other medical devices which are not implanted.

Non-limiting examples of implantable medical devices which may incorporate one or more CSA compounds, as described herein, include catheters, endotracheal tubes, intravenous feed lines, feeder tubes, drains, prosthesis components (e.g., voice prostheses), peristaltic pumps, tympanostomy tubes, tracheostomy tubes, and the like. At least some of the embodiments described herein are particularly advantageous in applications where device biofouling, device rejection, and associated infection and inflammation pathologies are common issues.

In some embodiments, an implantable medical device incorporates one or more CSA compounds by including a coating containing the one or more CSA compounds. For example, an implantable medical device may be coated with a hydrogel material including the one or more CSA compounds. In some embodiments, the hydrogel provides a lubricious coating to the medical device in addition to providing the beneficial functionality of the CSA compounds.

In some embodiments, an implantable medical device additionally or alternatively incorporates one or more CSA compounds by including the one or more CSA compounds within the structure of the medical device itself. For example, the one or more CSA compounds may be mixed with a moldable polymeric material prior to extruding or otherwise manipulating the material to form at least a portion of the implantable medical device. In this manner, the implantable medical device includes a reservoir of CSA compounds directly incorporated into the structure of the device.

The polymeric material of the medical device may be any polymeric material with suitable biological compatibility for the intended use of the finished implantable medical device. In presently preferred embodiments, the medical device is formed at least partially from a silicone, where the silicone has been mixed with the one or more CSA compounds such that the one or more CSA compounds are distributed within the silicone material.

Any of the CSA compounds described herein may be utilized for incorporation with an implantable medical device. In some embodiments, one or more CSA compounds are included in a salt form. Preferred salt forms include sulfuric acid addition salts or sulfonic acid addition salts, including NDSA addition salts such as 1,5-NDSA addition salts. These and other salt forms of CSAs have shown beneficial properties such as good flowability/mixability and storage stability. In particular, such salt forms of CSAs are useful for mixing with moldable polymeric materials to form a medical device having CSA compounds included within the structure of the medical device. For example, some salt forms of CSA compounds have been shown to have limited or no interaction with polymeric materials when mixed with the polymeric materials, leaving the CSA compounds in an active form capable of providing enhanced antimicrobial and/or anti-inflammatory functionality to the medical device formed from the polymeric materials.

Preferred embodiments are directed to implantable medical devices formed at least partly of silicone. Other medical device embodiments may include polyethylene, polypropylene, polystyrene, polyester, polycarbonate, polyvinyl chloride, polyacrylate, polysulfone, or combinations thereof.

Some embodiments of implantable medical devices incorporating one or more CSA compounds are directed toward endotracheal tubes (ETTs). In one embodiment, an ETT includes a hydrogel coating, where the hydrogel includes the one or more CSA compounds. The hydrogel coating may be applied to substantially the entire surface of the ETT. However, in preferred embodiments, the distal tip (e.g., distal most 1 cm, 3 cm, 5 cm, 10 cm, 15 cm) of the ETT is coated with the hydrogel coating to provide intended local effects without risking accidental extubation.

In some embodiments, the hydrogel coating reduces the coefficient of friction at the surface of the ETT (or other medical device to which it is applied) by up to about 5 times, 10 times, 15 times, 20 times, or 30 times. Frictional trauma to the mucosal tissue lining the trachea may allow bacteria to bypass the physical barrier normally crated by a contiguous tracheal mucosal lining. ETTs including a hydrogel coating beneficially prevent frictional damage to the tracheal mucosa, thereby reducing the transmission of microbes and/or inflammatory cytokines entering the lungs or crossing the blood/brain barrier.

Further, antimicrobial effects imparted by the CSA compounds incorporated into the ETT device act to reduce biofilm formation and associated occurrence of detrimental health issues (such as subglottic stenosis).

Embodiments of implantable medical devices described herein can provide a variety of benefits. For example, medical devices can have extended lifetimes as a result of reductions in fouling and biofilm formation. Some devices, such as tracheostomy tubes, are typically required for months at a time, but must be replaced as fouling occurs. Extending the usable life of such medical devices reduces costs and reduces patient trauma and medical risks associated with removing and replacing the device. Another example is a voice prosthesis. Such implants are intended to be permanent, yet they typically only last months at a time due to fungal and/or bacterial fouling.

One or more of the disclosed embodiments can reduce the occurrence of device-related infections, and thereby reduce the need for treatment with antibiotics or other antimicrobials. Further, the antimicrobial effects of such medical devices limit or reduce the need for prophylactic antibiotic administration. For example, antibiotics are typically administered prophylactically when tympanostomy tubes are implanted. A tympanostomy tube having one or more incorporated CSA compounds, as described herein, may reduce or eliminate the need to administer such antibiotics.

As explained in more detail below, one or more embodiments may be utilized to prevent or reduce: delirium, cognitive decline, and/or Alzheimer's disease in patients requiring mechanical ventilation; acute kidney injury in patients requiring mechanical ventilation; and prevent post extubation stridor and stenosis in mechanically ventilated patients.

### III. Methods of Manufacturing a Medical Device Incorporating CSA Compounds

In some embodiments, a method of manufacturing a medical device with one or more incorporated CSA compounds comprises: (1) providing a biologically compatible moldable polymeric material; (2) mixing one or more CSA compounds with the moldable polymeric material; and (3) molding the moldable polymeric material into an implantable medical device.

In some embodiments, the one or more CSA compounds are provided in salt form. In preferred embodiments, the one or more CSA compounds are provided in the form of a sulfonic acid addition salt, including disulfonic addition salts such as NDSA salt forms of CSAs. Such salt forms have shown to be flowable and readily mixable with polymeric materials to form the molded medical device structures. In addition, such salt forms have been shown to not react with or lose activity upon mixing with the polymeric materials, preserving the effectiveness of the CSA compounds in providing antimicrobial, anti-inflammatory, analgesic, and/or healing properties.

In some embodiments, the one or more CSA compounds are provided in a solid salt form. In some embodiments, solid form CSA compounds are processed to a desired average particle size prior to mixing with the moldable polymeric material, such as through a micronizing process using one or more impact mills (e.g., hammer mills, jet mills, and/or ball, pebble, or rod mills) or other suitable processing units. After sizing, the solid form CSA compounds will preferably have an average particle size of about 50 nm, 100 nm, 150 nm, 250 nm, 500 nm, 1 μm, or an average particle size within a range defined by any two of the foregoing values.

Medical devices manufactured so as to incorporate one or more CSA compounds within the structure of the medical device are particularly beneficial in applications in which the medical device is intended to be in use for long periods of time, and/or where microbial colonization and fouling is a likely problem. For example, where embodiments utilizing a coating of CSA compounds may have about 5-10 days of efficacy, certain embodiments incorporating one or more CSA compounds within the structure of the device have shown efficacy lasting at least about a month, with efficacy expected to endure for several months.

In preferred embodiments, the polymeric material includes silicone. Silicone has shown good mixability with at least some of the CSA compounds disclosed herein, with no indication of the silicone reacting with or reducing the activity of the CSA compounds.

One or more embodiments are directed to methods of manufacturing an implantable medical device, the method comprising: (1) providing an implantable medical device; and (2) applying a coating to at least a portion of a surface of the medical device to associate the coating with the medical device, the coating being formulated with one or more CSA compounds.

In preferred embodiments, the coating is a hydrogel formulated to provide the coating with lubricious properties. Hydrogels may be formed using one or more polymers such as polyvinyl alcohol, polyacrylic acid, polyethylene glycol, polyvinylpyrrolidone, polysaccharides, and polyacrylamide, for example. Hydrogels may be amorphous, semi-crystalline, or crystalline. In some embodiments, the hydrogel coating reduces the coefficient of friction at the surface of the medical device to which it is applied by up to about 5 times, 10 times, 15 times, 20 times, or 30 times.

### IV. Methods of Using a Medical Device Incorporating CSA Compounds

One or more embodiments are directed to methods of controlling microbial growth, including biofilm growth, on a medical device and/or at an implantation site at which an implantable medical device is implanted. In some embodiments, a method comprises: (1) implanting a medical device having one or more incorporated CSA compounds, and (2) the medical device killing one or more microbes contacting the implantable medical device. The implantable medical device may be effective in killing a wide variety of microbes. In some embodiments, the method provides enhanced protection from biofouling and/or associated infection (e.g., as compared to a similar implantable medical device not incorporating CSA compounds).

One or more embodiments are directed to methods of reducing inflammation at an implantation site at which a medical device is implanted. In some embodiments, a method comprises: (1) implanting a medical device having one or more incorporated CSA compounds, as described herein, and (2) the implantable medical device reducing or preventing inflammation at the treatment site (e.g., as compared to a similar implantable medical device not incorporating CSA compounds).

One or more embodiments are directed to methods of increasing the rate of tissue healing at an implantation site at which a medical device has been implanted. In some embodiments, a method comprises: (1) implanting a medical device having one or more incorporated CSA compounds, as described herein, and (2) the implantable medical device increasing the rate tissue healing at the implantation site (e.g., as compared to a similar implantable medical device not incorporating CSA compounds).

One or more of the methods described herein may be utilized to prevent or reduce post-extubation stridor and stenosis in mechanically ventilated patients. Such conditions are associated with excessive airway manipulation, traumatic intubation, and agitation during intubation. Without being bound to any particular mode of action, it is believed that embodiments of ETTs with one or more incorporated CSA compounds provide anti-inflammatory activity and/or tissue healing activity which reduce the effects of the intubation trauma associated with stridor and stenosis.

One or more of the methods described herein may be utilized to prevent or reduce delirium, cognitive decline, and/or Alzheimer's in patients requiring mechanical ventilation. Such conditions have been shown to be related to high serum levels of inflammatory cytokines, such as IL-6, TNF alpha, and others. These levels rise rapidly following intubation. Without being bound to any particular mode of action, it is believed that embodiments of ETTs having one or more incorporated CSA compounds provide anti-inflammatory activity and/or tissue healing activity which reduces or prevents cognitive decline associated with intubation. For example, CSA compounds have been shown to dampen the inflammatory response. Further, CSA compounds may promote faster healing of traumatized tracheal tissue and regeneration of a stable mucosal layer, thereby more quickly reducing pathways through which inflammatory cytokines can pass into systemic circulation. In addition, in embodiments in which an ETT includes a lubricious CSA coating material (e.g., hydrogel), the coating can reduce or prevent trauma to the tracheal tissues.

One or more of the methods described herein may be utilized to prevent or reduce acute kidney injury (AKI) in patients requiring mechanical ventilation. AKI can be a result of an inflammatory episode, and is associated with high serum levels of inflammatory cytokines. These levels can rise rapidly following intubation. Without being bound to any particular mode of action, it is believed that embodiments of ETTs having one or more incorporated CSA compounds provide anti-inflammatory activity and/or tissue healing activity which reduces or prevents cognitive decline associated with intubation. For example, CSA compounds have been shown to dampen the inflammatory response. Further, CSA compounds may promote faster healing of traumatized tracheal tissue and regeneration of a stable mucosal layer, thereby more quickly reducing pathways through which inflammatory cytokines can pass into systemic circulation. In addition, in embodiments in which an ETT includes a lubricious CSA coating material (e.g., hydrogel), the coating can reduce or prevent trauma to the tracheal tissues.

In some embodiments, the CSA compounds in the medical device maintain efficacy (for killing microbes, preventing or reducing inflammation, and/or accelerating wound healing) for at least 4 days after implantation, at least 7 days after implantation, at least 14 days after implantation, at least 30 days after implantation, at least 60 days after implantation, or about 90 days after implantation. In some embodiments, the medical device maintains efficacy for as long as the implant resides at the implantation site (e.g., about a weeks, about two weeks, about a month, about 2 or 3 months).

### V. Examples

### Example 1

To determine the role of synthetic Ceragenins CSA-13, 44 and 90 in inflammation using mesenchymal stem cells (MSC), targeted mRNA panels from SABiosciences, and primary cells from Lonza were selected. Cells were purchased from Lonza.com and used fresh for each test using recommended media and culture conditions. After treatment, mRNA was isolated using Qiagen RNeasy Mini Kit®, and quantified using a NanoDrop 2000® by UV at 260 nm and 260/280 ratio for purity. cDNA was made using a First Strand Kit® from SABiosciences and processed for real time PCR using a kit from the same company for selected analysis of wound healing pathways. Results from q-PCR were uploaded to the SABiosciences site and to Ingenuity.com web site for analysis and pathway mapping. On day 1, primary human MSC cells were plated at 200,000 cells/well using 6-well plates with 3 ml of recommended mediah—MSC Basal Medium+BulletKit (50 ml Growth Supplement, 10 ml L-Glutamine and 0.5 ml Gentamicin Sulfate Amphotercin-B) for 24 hours. Only early passages of cells were used, and never from frozen stock. On day 2, cells were treated with compounds dissolved in DMSO diluted 1:1000 or more to avoid effects of the solvent. Final testing concentration for CSA-13 was 5.0 μM. Treatment lasted 8 hours, and was followed by RNA isolation using QIAGEN RNeasy Mini Kit® (74104). RNA was measured at 260/280 nm using a NanoDrop 2000® and normalized to 2.4 ng per well, cDNA preparation was done using QIAGEN First Strand kit 330401. q-PCR was run as absolute quantification and threshold set at 0.1 units. Dendritic cells were plated at 500,000 cells/well using 24-well plate with 500 μl of Lonza LGM-3 Complete Growth Medium with and without compound. Treatment lasted 8 hours, and was followed by RNA isolation using QIAGEN RNeasy Mini Kit® (74104). RNA was measured at 260/280 nm using NanoDrop2000® and normalized to 2.4 ng per well, cDNA preparation was done using QIAGEN First Strand kit 330401. PCR was run as absolute quantification and threshold set at 0.1 units. The results of these experiments are summarized in Tables 1-3 for CSA-13, 44, and 90, respectively. The results highly the significant modulation of genes related to inflammation, such as IL1A (Interleukin-1 alpha), IL1B (Interleukin-1 beta), TLR2 (Toll-like receptor 2), TLR4 (Toll-like receptor 4), TLR6 (Toll-like receptor 6), TLR8 (Toll-like receptor 8), TLR9 (Toll-like receptor 9), TNF (Tumor necrosis factor), TNFRSF1A (Tmor necrosis factor receptor superfamily member 1A), IRAK2 (Interleukin-1 receptor-associated kinase 2), NFKB1 (Nuclear factor of kappa light polypeptide gene enhancer in B-cells 1), NFKB2 (Nuclear factor of kappa light polypeptide gene enhancer in B-cells 2), and NFKBIA (Nuclear factor of kappa light polypeptide gene enhancer in B-cells inhibitor, alpha). Such results clearly illustrate the potential of CSAs for modulating inflammation.

TABLE 1
Gene Expression Results for CSA-13

| Gene Symbol | Fold Regulation |
|---|---|
| IL1A | −5.5237 |
| IL1B | −16.3901 |
| TLR2 | −7.6418 |
| TLR4 | −2.6139 |
| TLR6 | −4.8417 |
| TLR8 | −2.107 |
| TLR9 | −2.1421 |
| TNF | −8.1805 |
| TNFRSF1A | −5.1031 |
| IRAK2 | −43.5175 |
| NFKB1 | −3.4437 |
| NFKB2 | −4.2155 |
| NFKBIA | −22.966 |

TABLE 2
Gene Expression Results for CSA-44

| Gene Symbol | Fold Regulation |
|---|---|
| IL1A | −6.0325 |
| IL1B | −28.5329 |
| IRAK2 | −31.8021 |
| NFKB1 | −3.2891 |
| NFKB2 | −2.2766 |
| NFKBIA | −52.206 |
| TLR2 | −15.7179 |
| TLR4 | −2.977 |
| TLR6 | −2.392 |
| TLR8 | −8.2256 |
| TLR9 | −1.8905 |
| TNF | −25.9588 |
| TNFRSF1A | −2.2461 |

TABLE 3
Gene Expression Results for CSA-90

| Gene Symbol | Fold Regulation |
|---|---|
| IL1A | −6.96 |
| 1L1B | −3.6734 |
| IRAK2 | −52.0069 |
| NFKB1 | −4.718 |
| NFKB2 | −2.5474 |
| NFKBIA | −26.0352 |
| TLR2 | −13.6933 |
| TLR4 | −3.4278 |
| TLR6 | −2.0885 |
| TLR8 | −4.1972 |
| TLR9 | −1.8613 |
| TNF | −4.8514 |
| TNFRSF1A | −7.3196 |

### Example 2

IL-6 is a marker of systemic inflammation. Female C57/BL6 mice were infected in the respiratory tract with a non-lethal dose of P. aeruginosa as a model of pneumonia. One cohort (n=6) also received 80 mg/kg CSA-13; a second cohort (n=6) also received 40 mg/kg CSA-13; a third (n=6) received no CSA treatment; and a fourth (n=6) was not infected. Examination of IL-6 levels in the kidneys 24 hours post-infection demonstrated that those infected animals not treated with CSA had IL-6 levels>15 times those of control and 5-10 times higher than those of the CSA-treated animals. Thus, treatment with CSA significantly reduced kidney IL-6 levels in a pneumonia model.

### Example 3

A silicone-based Foley catheter was coated with a hydrogel coating of approximately 10 μm in thickness. The coating included CSA-131. The coating was initially shown to maintain efficacy for about 6 or 7 days.

### Example 4

A silicone-based Foley catheter was formed using silicone mixed with an NDSA salt form of CSA-131. The silicone catheter was shown to maintain high efficacy for the first three weeks, with test data showing efficacy lasting for at least 3 or 4 months.

### Example 5

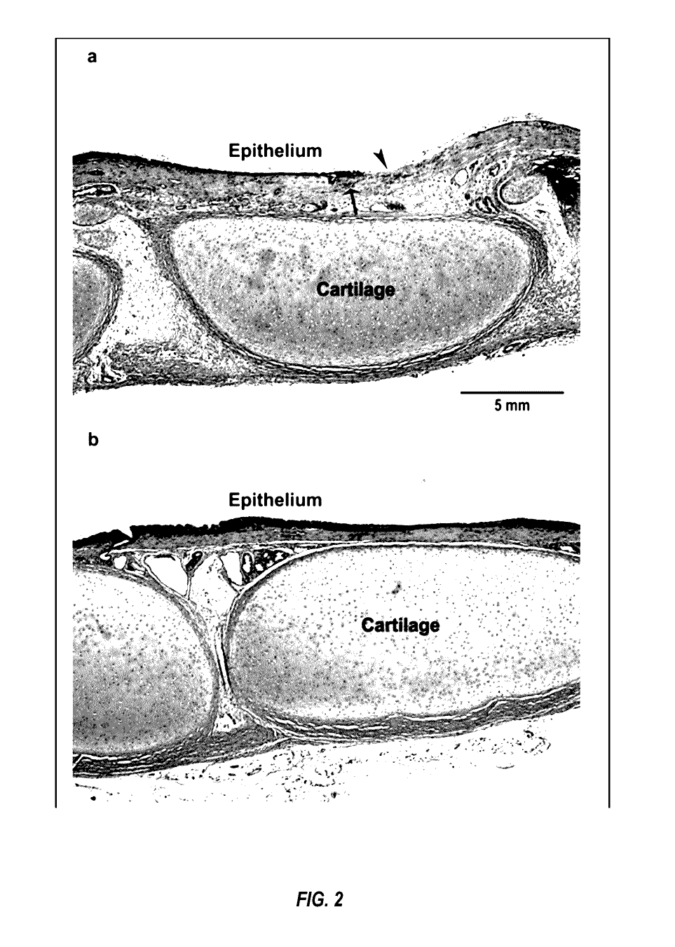

Pre-term lambs were intubated using ETTs including a coating having CSA-131 and tracheal mucosal integrity of the lambs was compared to the tracheal mucosal integrity of a control group (intubated with uncoated ETTs). The pre-term lambs intubated with coated ETTs showed markedly improved mucosal integrity compared to the pre-term lambs of the control group. FIG. 2 illustrates the histological appearance of tracheas of the premature lambs that were intubated for three days. Image (a) shows a trachea from a lamb intubated with an uncoated ETT. An area of denuded epithelium (arrowhead), and accumulation of white blood cells (arrow) are highlighted. Image (b) shows a trachea from a lamb intubated with an ETT coated with a CSA-131 containing coating. As shown, the epithelium is intact and the subjacent connective tissue region is not inflamed.

### VI. Additional Details of CSA Compounds

More specific examples of CSA compounds according to Formula I are shown below in Formulas II and III, wherein Formula III differs from Formula II by omitting R₁₅ and the ring carbon to which it is attached. The R groups shown in the Formulae can have a variety of different structures. CSA compounds, and a variety of different R groups, useful in accordance with the present disclosure, are disclosed in U.S. Pat. Nos. 6,350,738, 6,486,148, 6,767,904, 7,598,234, and 7,754,705, which are incorporated herein by reference.

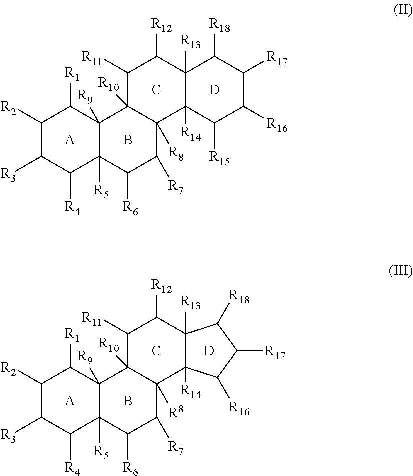

In some embodiments of Formulas II and III, at least two of R₃, R₇, and R₁₂ may independently include a cationic moiety (e.g., amino or guanidino groups) bonded to the steroid backbone structure via a non-hydrolysable or hydrolysable linkage. For the embodiments of the present disclosure, the linkage is preferably non-hydrolysable under conditions of sterilization and storage, and physiological conditions. Such cationic functional groups (e.g., amino or guanidino groups) may be separated from the backbone by at least one, two, three, four or more atoms.

Optionally, a tail moiety may be attached to the backbone structures at R₁₈. The tail moiety may have variable chain length or size and may be charged, uncharged, polar, non-polar, hydrophobic, amphipathic, and the like. The tail moiety may, for example, be configured to alter the hydrophobicity/hydrophilicity of the ceragenin compound. CSA compounds of the present disclosure having different degrees of hydrophobicity/hydrophilicity may, for example, have different rates of uptake into different target microbes.

The R groups described herein, unless specified otherwise, may be substituted or unsubstituted.

In some embodiments shown by Formulas II and III:

each of fused rings A, B, C, and D may be independently saturated, or may be fully or partially unsaturated, provided that at least two of A, B, C, and D are saturated, wherein rings A, B, C, and D form a ring system. Other ring systems can also be used, e.g., 5-member fused rings and/or compounds with backbones having a combination of 5- and 6-membered rings;

R₁ through R₄, R₆, R₇, R₁₁, R₁₂, R₁₅, R₁₆, and R₁₈ are independently selected from the group consisting of hydrogen, hydroxyl, alkyl, hydroxyalkyl, alkyloxyalkyl, alkylcarboxyalkyl, alkylaminoalkyl, alkylaminoalkylamino, alkylaminoalkylamino-alkylamino, aminoalkyl, aryl, arylaminoalkyl, haloalkyl, alkenyl, alkynyl, oxo, a linking group attached to a second steroid, aminoalkyloxy, aminoalkyloxyalkyl, aminoalkylcarboxy, aminoalkylaminocarbonyl, aminoalkylcarboxamido, di(alkyl)aminoalkyl, H₂N—HC(Q₅)-C(O)—O—, H₂N—HC(Q₅)-C(O)—N(H)—, azidoalkyloxy, cyanoalkyloxy, P.G.-HN—HC(Q₅)-C(O)—O—, guanidinoalkyloxy, quaternary ammonium alkylcarboxy, and guanidinoalkyl carboxy, where Q₅ is a side chain of any amino acid (including a side chain of glycine, i.e., H), and P.G. is an amino protecting group; and

R₅, R₈, R₉, R₁₀, R₁₃, R₁₄ and R₁₈ are independently deleted when one of rings A, B, C, or D is unsaturated so as to complete the valency of the carbon atom at that site, or R₅, R₈, R₉, R₁₀, R₁₃, and R₁₄ are independently selected from the group consisting of hydrogen, hydroxyl, alkyl, hydroxyalkyl, alkyloxyalkyl, aminoalkyl, aryl, haloalkyl, alkenyl, alkynyl, oxo, a linking group attached to a second steroid, aminoalkyloxy, aminoalkylcarboxy, aminoalkylaminocarbonyl, di(alkyl)aminoalkyl, H₂N—HC(Q₅)-C(O)—O—, H₂N—HC(Q₅)-C(O)—N(H)—, azidoalkyloxy, cyanoalkyloxy, P.G.-HN—HC(Q₅)-C(O)—O—, guanidinoalkyloxy, and guanidinoalkyl-carboxy, where Q₅ is a side chain of any amino acid, P.G. is an amino protecting group.

In some embodiments, at least one, and sometimes two or three of R₁₋₄, R₆, R₇, R₁₁, R₁₂, R₁₅, R₁₆, R₁₇, and R₁₈ are independently selected from the group consisting of aminoalkyl, aminoalkyl oxy, alkylcarboxyalkyl, alkyl aminoalkyl amino, alkyl aminoalkyl-aminoalkylamino, aminoalkylcarboxy, arylaminoalkyl, aminoalkyloxyaminoalkylamino-carbonyl, aminoalkylaminocarbonyl, aminoalkyl-carboxyamido, a quaternary ammonium alkylcarboxy, di(alkyl)aminoalkyl, H₂N—HC(Q₅)-C(O)—O—, H₂N—HC(Q₅)-C(O)—N(H)—, azidoalkyloxy, cyanoalkyloxy, P.G.-HN—HC(Q₅)-C(O)—O—, guanidine-alkyloxy, and guanidinoalkylcarboxy.

In some embodiments, R₁ through R₄, R₆, R₇, R₁₁, R₁₂, R₁₅, R₁₆, and R₁₈ are independently selected from the group consisting of hydrogen, hydroxyl, (C₁-C₂₂) alkyl, (C₁-C₂₂) hydroxyalkyl, (C₁-C₂₂) alkyloxy-(C₁-C₂₂) alkyl, (C₁-C₂₂) alkylcarboxy-(C₁-C₂₂) alkyl, (C₁-C₂₂) alkylamino-(C₁-C₂₂) alkyl, (C₁-C₂₂) alkylamino-(C₁-C₂₂) alkylamino, (C₁-C₂₂) alkylamino-(C₁-C₂₂) alkylamino-(C₁-C₂₂) alkylamino, (C₁-C₂₂) aminoalkyl, aryl, arylamino-(C₁C₂₂) alkyl, (C₁-C₂₂) haloalkyl, C₂-C₆ alkenyl, C₂-C₆ alkynyl, oxo, a linking group attached to a second steroid, (C₁-C₂₂) aminoalkyloxy, (C₁-C₂₂) aminoalkyloxy-(C₁-C₂₂) alkyl, (C₁-C₂₂) aminoalkylcarboxy, (C₁-C₂₂) aminoalkylaminocarbonyl, (C₁-C₂₂) aminoalkyl-carboxamido, di(C₁-C₂₂ alkyl)aminoalkyl, H₂N—HC(Q₅)-C(O)—O—, H₂N—HC(Q₅)-C(O)—N(H)—, (C₁-C₂₂) azidoalkyloxy, (C₁-C₂₂) cyanoalkyloxy, P.G.-HN—HC(Q₅)-C(O)—O—, (C₁-C₂₂) guanidinoalkyloxy, (C₁-C₂₂) quaternary ammonium alkylcarboxy, and (C₁-C₂₂) guanidinoalkyl carboxy, where Q₅ is a side chain of an amino acid (including a side chain of glycine, i.e., H), and P.G. is an amino protecting group; and

R₅, R₈, R₉, R₁₀, R₁₃, R₁₄ and R₁₇ are independently deleted when one of rings A, B, C, or D is unsaturated so as to complete the valency of the carbon atom at that site, or R₅, R₈, R₉, R₁₀, R₁₃, and R₁₄ are independently selected from the group consisting of hydrogen, hydroxyl, (C₁-C₂₂) alkyl, (C₁-C₂₂) hydroxyalkyl, (C₁-C₂₂) alkyloxy-(C₁-C₂₂) alkyl, (C₁-C₂₂) aminoalkyl, aryl, (C₁-C₂₂) haloalkyl, (C₂-C₆) alkenyl, (C₂-C₆) alkynyl, oxo, a linking group attached to a second steroid, (C₁-C₂₂) aminoalkyloxy, (C₁-C₂₂) aminoalkylcarboxy, (C₁-C₂₂) aminoalkylaminocarbonyl, di(C₁-C₂₂ alkyl)aminoalkyl, H₂N—HC(Q₅)-C(O)—O—, H₂N—HC(Q₅)-C(O)—N(H)—, (C₁-C₂₂) azidoalkyloxy, (C₁-C₂₂) cyanoalkyloxy, P.G.-HN—HC(Q₅)-C(O)—O—, (C₁-C₂₂) guanidinoalkyloxy, and (C₁-C₂₂) guanidinoalkylcarboxy, where Q5 is a side chain of any amino acid, and P.G. is an amino protecting group;

provided that at least two or three of R₁₋₄, R₆, R₇, R₁₁, R₁₂, R₁₅, R₁₆, R₁₇, and R₁₈ are independently selected from the group consisting of (C₁-C₂₂) aminoalkyl, (C₁-C₂₂) aminoalkyloxy, (C₁-C₂₂) alkylcarboxy-(C₁-C₂₂) alkyl, (C₁-C₂₂) alkylamino-(C₁-C₂₂) alkylamino, (C₁-C₂₂) alkylamino-(C₁-C₂₂) alkylamino (C₁-C₂₂) alkylamino, (C₁-C₂₂) aminoalkylcarboxy, arylamino (C₁-C₂₂) alkyl, (C₁-C₂₂) aminoalkyloxy (C₁-C₂₂) aminoalkylaminocarbonyl, aminoalkylaminocarbonyl, (C₁-C₂₂) (C₁-C₂₂) aminoalkylcarboxyamido, (C₁-C₂₂) quaternary ammonium alkylcarboxy, di(C₁-C₂₂ alkyl)aminoalkyl, H₂N—HC(Q₅)-C(O)—O—, H₂N—HC(Q₅)-C(O)—N(H)—, (C₁-C₂₂) azidoalkyloxy, (C₁-C₂₂) cyanoalkyloxy, P.G.-HN—HC(Q₅)-C(O)—O—, (C₁-C₂₂) guanidinoalkyloxy, and (C₁-C₂₂) guanidinoalkylcarboxy.

In some embodiments, R₁ through R₄, R₆, R₇, R₁₁, R₁₂, R₁₅, R₁₆, and R₁₈ are independently selected from the group consisting of hydrogen, hydroxyl, (C₁-C₁₈) alkyl, (C₁-C₁₈) hydroxyalkyl, (C₁-C₁₈) alkyloxy-(C₁-C₁₈) alkyl, (C₁-C₁₈) alkylcarboxy-(C₁-C₁₈) alkyl, (C₁-C₁₈) alkylamino-(C₁-C₁₈)alkyl, (C₁-C₁₈) alkylamino-(C₁-C₁₈) alkylamino, (C₁-C₁₈) alkylamino-(C₁-C₁₈) alkylamino-(C₁-C₁₈) alkylamino, (C₁-C₁₈) aminoalkyl, aryl, arylamino-(C₁-C₁₈) alkyl, oxo, (C₁-C₁₈) aminoalkyloxy, (C₁-C₁₈) aminoalkyloxy-(C₁-C₁₈) alkyl, (C₁-C₁₈) aminoalkylcarboxy, (C₁-C₁₈) aminoalkylaminocarbonyl, (C₁-C₁₈) aminoalkyl-carboxamido, di(C₁-C₁₈ alkyl)aminoalkyl, (C₁-C₁₈) guanidinoalkyloxy, (C₁-C₁₈) quaternary ammonium alkylcarboxy, and (C₁-C₁₈) guanidinoalkyl carboxy; and

R₅, R₈, R₉, R₁₀, R₁₃, R₁₄ and R₁₇ are independently deleted when one of rings A, B, C, or D is unsaturated so as to complete the valency of the carbon atom at that site, or R₅, R₈, R₉, R₁₀, R₁₃, and R₁₄ are independently selected from the group consisting of hydrogen, hydroxyl, (C₁-C₁₈) alkyl, (C₁-C₁₈) hydroxyalkyl, (C₁-C₁₈) alkyloxy-(C₁-C₁₈) alkyl, (C₁-C₁₈) alkylcarboxy-(C₁-C₁₈) alkyl, (C₁-C₁₈) alkylamino-(C₁-C₁₈)alkyl, (C₁-C₁₈) alkylamino-(C₁-C₁₈) alkylamino, (C₁-C₁₈) alkylamino-(C₁-C₁₈) alkylamino-(C₁-C₁₈) alkylamino, (C₁-C₁₈) aminoalkyl, aryl, arylamino-(C₁-C₁₈) alkyl, oxo, (C₁-C₁₈) aminoalkyloxy, (C₁-C₁₈) aminoalkyloxy-(C₁-C₁₈) alkyl, aminoalkylcarboxy (C₁-C₁₈), aminoalkylaminocarbonyl, (C₁-C₁₈) aminoalkylcarboxamido, di(C₁-C₁₈ alkyl)aminoalkyl, (C₁-C₁₈) guanidinoalkyloxy, (C₁-C₁₈) quaternary ammonium alkylcarboxy, and (C₁-C₁₈) guanidinoalkyl carboxy,

provided that at least two or three of R₁₋₄, R₆, R₇, R₁₁, R₁₂, R₁₅, R₁₆, R₁₇, and R₁₈ are independently selected from the group consisting of hydrogen, hydroxyl, an unsubstituted (C₁-C₁₈) alkyl, unsubstituted (C₁-C₁₈) hydroxyalkyl, unsubstituted (C₁-C₁₈) alkyloxy-(C₁-C₁₈) alkyl, unsubstituted (C₁-C₁₈) alkylcarboxy-(C₁-C₁₈) alkyl, unsubstituted (C₁-C₁₈) alkylamino-unsubstituted (C₁-C₁₈) alkylamino-(C₁-C₁₈) alkylamino, unsubstituted (C₁-C₁₈) alkylamino-(C₁-C₁₈) alkylamino-alkylamino, an unsubstituted (C₁-C₁₈) aminoalkyl, an unsubstituted aryl, an unsubstituted arylamino-(C₁-C₁₈) alkyl, oxo, an unsubstituted (C₁-C₁₈) aminoalkyloxy, an unsubstituted (C₁-C₁₈) aminoalkyloxy-(C₁-C₁₈) alkyl, an unsubstituted (C₁-C₁₈) aminoalkylcarboxy, an unsubstituted (C₁-C₁₈) aminoalkylaminocarbonyl, an unsubstituted (C₁-C₁₈) aminoalkylcarboxamido, an unsubstituted di(C₁-C₁₈ alkyl)aminoalkyl, unsubstituted (C₁-C₁₈) guanidinoalkyloxy, unsubstituted (C₁-C₁₈) quaternary ammonium alkylcarboxy, and unsubstituted (C₁-C₁₈) guanidinoalkyl carboxy.

In some embodiments, R₃, R₇, R₁₂, and R₁₈ are independently selected from the group consisting of hydrogen, an unsubstituted (C₁-C₁₈) alkyl, unsubstituted hydroxyalkyl, unsubstituted (C₁-C₁₈) alkyl oxy-(C₁-C₁₈) alkyl, unsubstituted alkylcarboxy-(C₁-C₁₈) alkyl, unsubstituted (C₁-C₁₈) alkylamino-(C₁-C₁₈)alkyl, unsubstituted (C₁-C₁₈) alkylamino-(C₁-C₁₈) alkylamino, unsubstituted (C₁-C₁₈) alkylamino-(C₁-C₁₈) alkylamino-(C₁-C₁₈) alkylamino, an unsubstituted (C₁-C₁₈) aminoalkyl, an unsubstituted arylamino-(C₁-C₁₈) alkyl, an unsubstituted (C₁-C₁₈) aminoalkyloxy, an unsubstituted (C₁-C₁₈) aminoalkyloxy-(C₁-C₁₈) alkyl, an unsubstituted (C₁-C₁₈) aminoalkylcarboxy, an unsubstituted (C₁-C₁₈) aminoalkylaminocarbonyl, an unsubstituted (C₁-C₁₈) aminoalkylcarboxamido, an unsubstituted di(C₁-C₁₈ alkyl)aminoalkyl, unsubstituted (C₁-C₁₈) guanidinoalkyloxy, unsubstituted (C₁-C₁₈) quaternary ammonium alkylcarboxy, and unsubstituted (C₁-C₁₈) guanidinoalkyl carboxy.

In some embodiments, R₁, R₂, R₄, R₅, R₆, R₈, R₉, R₁₀, R₁₁, R₁₃, R₁₄, R₁₅, R₁₆, and R₁₇ are independently selected from the group consisting of hydrogen and unsubstituted (C₁-C₆) alkyl.

In some embodiments, R₃, R₇, R₁₂, and R₁₈ are independently selected from the group consisting of hydrogen, an unsubstituted (C₁-C₆) alkyl, unsubstituted (C₁-C₆) hydroxyalkyl, unsubstituted (C₁-C₁₆) alkyloxy-(C₁-C₅) alkyl, unsubstituted (C₁-C₁₆) alkylcarboxy-(C₁-C₅) alkyl, unsubstituted (C₁-C₁₆) alkylamino-(C₁-C₅)alkyl, (C₁-C₁₆) alkylamino-(C₁-C₅) alkylamino, unsubstituted (C₁-C₁₆) alkylamino-(C₁-C₁₆) alkylamino-(C₁-C₅) alkylamino, an unsubstituted (C₁-C₁₆) aminoalkyl, an unsubstituted arylamino-(C₁-C₅) alkyl, an unsubstituted (C₁-C₅) aminoalkyloxy, an unsubstituted (C₁-C₁₆) aminoalkyloxy-(C₁-C₅) alkyl, an unsubstituted (C₁-C₅) aminoalkylcarboxy, an unsubstituted (C₁-C₅) aminoalkylaminocarbonyl, an unsubstituted (C₁-C₅) aminoalkylcarboxamido, an unsubstituted di(C₁-C₅ alkyl)amino-(C₁-C₅) alkyl, unsubstituted (C₁-C₅) guanidinoalkyloxy, unsubstituted (C₁-C₁₆) quaternary ammonium alkylcarboxy, and unsubstituted (C₁-C₁₆) guanidinoalkylcarboxy.

In some embodiments, R₁, R₂, R₄, R₅, R₆, R₈, R₁₀, R₁₁, R₁₄, R₁₆, and R₁₇ are each hydrogen; and R₉ and R₁₃ are each methyl.

In some embodiments, R₃, R₇, R₁₂, and R₁₈ are independently selected from the group consisting of aminoalkyloxy; aminoalkylcarboxy; alkylaminoalkyl; alkoxycarbonylalkyl; alkylcarbonyl alkyl; di(alkyl)aminoalkyl; alkylcarboxyalkyl; and hydroxyalkyl.

In some embodiments, R₃, R₇, and R₁₂ are independently selected from the group consisting of aminoalkyloxy and aminoalkylcarboxy; and R₁₈ is selected from the group consisting of alkylaminoalkyl; alkoxycarbonylalkyl; alkylcarbonyloxyalkyl; di(alkyl)aminoalkyl; alkylaminoalkyl; alkyoxycarbonylalkyl; alkylcarboxyalkyl; and hydroxyalkyl.

In some embodiments, R₃, R₇, and R₁₂ are the same.

In some embodiments, R₃, R₇, and R₁₂ are aminoalkyloxy.

In some embodiments, R₁₈ is alkylaminoalkyl.

In some embodiments, R₁₈ is alkoxycarbonylalkyl.

In some embodiments, R₁₈ is di(alkyl)aminoalkyl.

In some embodiments, R₁₈ is alkylcarboxyalkyl.

In some embodiments, R₁₈ is hydroxyalkyl.

In some embodiments, R₃, R₇, and R₁₂ are aminoalkylcarboxy.

In some embodiments, R₃, R₇, R₁₂, and R₁₈ are independently selected from the group consisting of aminoalkyloxy; aminoalkylcarboxy; alkylaminoalkyl; di-(alkyl)aminoalkyl; alkoxycarbonylalkyl; and alkylcarboxyalkyl.

In some embodiments, R₃, R₇, and R₁₂ are independently selected from the group consisting of aminoalkyloxy and aminoalkylcarboxy, and wherein R₁₈ is selected from the group consisting of alkylaminoalkyl; di-(alkyl)aminoalkyl; alkoxycarbonylalkyl; and alkylcarboxyalkyl.

In some embodiments, R₃, R₇, and R₁₂ are independently selected from the group consisting of aminoalkyloxy and aminoalkylcarboxy, and wherein R₁₈ is selected from the group consisting of alkylaminoalkyl; di-(alkyl)aminoalkyl; and alkoxycarbonylalkyl.

In some embodiments, R₃, R₇, R₁₂, and R₁₈ are independently selected from the group consisting of amino-C₃-alkyl oxy; amino-C₃-alkyl-carboxy; C₈-alkyl amino-C₅-alkyl; C₁₂-alkylamino-C₅-alkyl; C₁₃-alkylamino-C₅-alkyl; C₁₆-alkylamino-C₅-alkyl; di-(C₅-alkyl)amino-C₅-alkyl; C₆-alkoxy-carbonyl-C₄-alkyl; C₈-alkoxy-carbonyl-C₄-alkyl; C₁₀-alkoxy-carbonyl-C₄-alkyl; C₆-alkyl-carboxy-C₄-alkyl; C₈-alkyl-carboxy-C₄-alkyl; and C₁₀-alkyl-carboxy-C₄-alkyl.

In some embodiments, R₃, R₇, R₁₂, and R₁₈ are independently selected from the group consisting of amino-C₃-alkyloxy; amino-C₃-alkyl-carboxy; C₈-alkylamino-C₅-alkyl; C₁₂-alkylamino-C₅-alkyl; C₁₃-alkylamino-C₅-alkyl; C₁₆-alkylamino-C₅-alkyl; di-(C₅-alkyl)amino-C₅-alkyl; C₆-alkoxy-carbonyl-C₄-alkyl; C₈-alkoxy-carbonyl-C₄-alkyl; and C₁₀-alkoxy-carbonyl-C₄-alkyl.

In some embodiments, R₃, R₇, and R₁₂, are independently selected from the group consisting of amino-C₃-alkyloxy or amino-C₃-alkyl-carboxy, and wherein R₁₈ is selected from the group consisting of C₈-alkylamino-C₅-alkyl; C₁₂-alkylamino-C₅-alkyl; C₁₃-alkylamino-C₅-alkyl; C₁₆-alkyl amino-C₅-alkyl; di-(C₅-alkyl)amino-C₅-alkyl; C₆-alkoxy-carbonyl-C₄-alkyl; C₈-alkoxy-carbonyl-C₄-alkyl; C₁₀-alkoxy-carbonyl-C₄-alkyl; C₆-alkyl-carboxy-C₄-alkyl; C₈-alkyl-carboxy-C₄-alkyl; and C₁₀-alkyl-carboxy-C₄-alkyl.

In some embodiments, R₃, R₇, and R₁₂, are independently selected from the group consisting of amino-C₃-alkyloxy or amino-C₃-alkyl-carboxy, and wherein R₁₈ is selected from the group consisting of C₈-alkylamino-C₅-alkyl; C₁₂-alkylamino-C₅-alkyl; C₁₃-alkylamino-C₅-alkyl; C₁₆-alkyl amino-C₅-alkyl; di-(C₅-alkyl)amino-C₅-alkyl; C₆-alkoxy-carbonyl-C₄-alkyl; C₈-alkoxy-carbonyl-C₄-alkyl; and C₁₀-alkoxy-carbonyl-C₄-alkyl.

In some embodiments, R₃, R₇, R₁₂, and R₁₈ are independently selected from the group consisting of amino-C₃-alkyloxy; amino-C₃-alkyl-carboxy; amino-C₂-alkylcarboxy; C₈-alkylamino-C₅-alkyl; C₈-alkoxy-carbonyl-C₄-alkyl; C₁₀-alkoxy-carbonyl-C₄-alkyl; C₈-alkyl-carbonyl-C₄-alkyl; di-(C₅-alkyl)amino-C₅-alkyl; C₁₃-alkylamino-C₅-alkyl; C₆-alkoxy-carbonyl-C₄-alkyl; C₆-alkyl-carboxy-C₄-alkyl; C₁₆-alkylamino-C₅-alkyl; C₁₂-alkylamino-C₅-alkyl; and hydroxy(C₅)alkyl.

In some embodiments, R₁₈ is selected from the group consisting of C₈-alkylamino-C₅-alkyl or C₈-alkoxy-carbonyl-C₄-alkyl.

In some embodiments, at least R₁₈ can have the following structure:

—R₂₀—(C═O)—N—R₂₁R₂₂

wherein R₂₀ is omitted or alkyl, alkenyl, alkynyl, or aryl, and R₂₁ and R₂₂ are independently selected from the group consisting of hydrogen, alkyl, alkenyl, alkynyl, or aryl, provided that at least one of R₂₁ and R₂₂ is not hydrogen.

In some embodiments, R₂₁ and R₂₂ are independently selected from the group consisting of hydrogen, C₁-C₂₄ alkyl, C₂-C₂₄ alkenyl, C₂-C₂₄ alkynyl, C₆ or C₁₀ aryl, 5 to 10 membered heteroaryl, 5 to 10 membered heterocyclyl, C₇₋₁₃ aralkyl, (5 to 10 membered heteroaryl)-C₁-C₆ alkyl, C₃₋₁₀ carbocyclyl, C₄₋₁₀ (carbocyclyl)alkyl, (5 to 10 membered heterocyclyl)-C₁-C₆ alkyl, amido, and a suitable amine protecting group, provided that at least one of R₂₁ and R₂₂ is not hydrogen. In some embodiments, R₂₁ and R₂₂, together with the atoms to which they are attached, form a 5 to 10 membered heterocyclyl ring.

In some embodiments, one or more of rings A, B, C, and D are heterocyclic.

In some embodiments, rings A, B, C, and D are non-heterocyclic.

In some embodiments, the CSA compound is a compound of Formula IV, which is a subset of Formula III, or salt thereof, having a steroidal backbone:

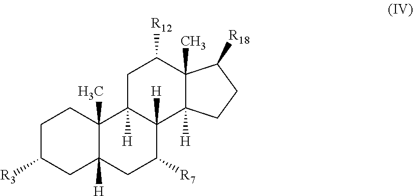

In some embodiments, R₃, R₇, and R₁₂ are independently selected from the group consisting of hydrogen, an unsubstituted (C₁-C₂₂) alkyl, unsubstituted (C₁-C₂₂) hydroxyalkyl, unsubstituted (C₁-C₂₂) alkyloxy-(C₁-C₂₂) alkyl, unsubstituted (C₁-C₂₂) alkylcarboxy-(C₁-C₂₂) alkyl, unsubstituted (C₁-C₂₂) alkyl amino-(C₁-C₂₂)alkyl, unsubstituted (C₁-C₂₂) alkyl amino-(C₁-C₂₂) alkylamino, unsubstituted (C₁-C₂₂) alkylamino-(C₁-C₂₂) alkylamino-(C₁-C₁₈) alkylamino, an unsubstituted (C₁-C₂₂) aminoalkyl, an unsubstituted arylamino-(C₁-C₂₂) alkyl, an unsubstituted (C₁-C₂₂) aminoalkyloxy, an unsubstituted (C₁-C₂₂) aminoalkyloxy-(C₁-C₂₂) alkyl, an unsubstituted (C₁-C₂₂) aminoalkylcarboxy, an unsubstituted (C₁-C₂₂) aminoalkyl-aminocarbonyl, an unsubstituted (C₁-C₂₂) aminoalkylcarboxamido, an unsubstituted di(C₁-C₂₂ alkyl)aminoalkyl, unsubstituted (C₁-C₂₂) guanidinoalkyloxy, unsubstituted (C₁-C₂₂) quaternary ammonium alkylcarboxy, and unsubstituted (C₁-C₂₂) guanidinoalkylcarboxy.

In some embodiments, R₃, R₇, and R₁₂ are independently selected from the group consisting of hydrogen, an unsubstituted (C₁-C₆) alkyl, unsubstituted (C₁-C₆) hydroxyalkyl, unsubstituted (C₁-C₁₆) alkyloxy-(C₁-C₅) alkyl, unsubstituted (C₁-C₁₆) alkylcarboxy-(C₁-C₅) alkyl, unsubstituted (C₁-C₁₆) alkyl amino-(C₁-C₅)alkyl, unsubstituted (C₁-C₁₆) alkyl amino-(C₁-C₅) alkylamino, unsubstituted (C₁-C₁₆) alkylamino-(C₁-C₁₆) alkylamino-(C₁-C₅) alkylamino, an unsubstituted (C₁-C₁₆) aminoalkyl, an unsubstituted arylamino-(C₁-C₅) alkyl, an unsubstituted (C₁-C₅) aminoalkyloxy, an unsubstituted (C₁-C₁₆) aminoalkyloxy-(C₁-C₅) alkyl, an unsubstituted (C₁-C₅) aminoalkylcarboxy, an unsubstituted (C₁-C₅) aminoalkylaminocarbonyl, an unsubstituted (C₁-C₅) aminoalkylcarboxamido, an unsubstituted di(C₁-C₅ alkyl)amino-(C₁-C₅) alkyl, unsubstituted (C₁-C₅) guanidinoalkyloxy, unsubstituted (C₁-C₁₆) quaternary ammonium alkylcarboxy, and unsubstituted (C₁-C₁₆) guanidinoalkylcarboxy.

In some embodiments, R₃, R₇, and R₁₂ are independently selected from the group consisting of aminoalkyloxy; aminoalkylcarboxy; alkylaminoalkyl; alkoxycarbonylalkyl; alkylcarbonylalkyl; di(alkyl)aminoalkyl; alkylcarboxyalkyl; and hydroxyalkyl.

In some embodiments, R₃, R₇, and R₁₂ are independently selected from the group consisting of aminoalkyloxy and aminoalkylcarboxy.

In some embodiments, R₃, R₇, and R₁₂ are the same. In some embodiments, R₃, R₇, and R₁₂ are aminoalkyloxy. In some embodiments, R₃, R₇, and R₁₂ are aminoalkylcarboxy.

In some embodiments, R₃, R₇, and R₁₂ are independently selected from the group consisting of amino-C₃-alkyloxy; amino-C₃-alkyl-carboxy; C₈-alkylamino-C₅-alkyl; C₈-alkoxy-carbonyl-C₄-alkyl; C₈-alkyl-carbonyl-C₄-alkyl; di-(C₅-alkyl)amino-C₅-alkyl; C₁₃-alkylamino-C₅-alkyl; C₆-alkoxy-carbonyl-C₄-alkyl; C₆-alkyl-carboxy-C₄-alkyl; and C₁₆-alkylamino-C₅-alkyl.

In some embodiments, CSA compounds as disclosed herein can be a compound of Formula I, Formula II, Formula III, Formula IV, or salts thereof wherein at least R₁₈ of the steroidal backbone includes amide functionality in which the carbonyl group of the amide is positioned between the amido nitrogen of the amide and fused ring D of the steroidal backbone. For example, any of the embodiments described above can substitute R₁₈ for an R₁₈ including amide functionality in which the carbonyl group of the amide is positioned between the amido nitrogen of the amide and fused ring D of the steroidal backbone.

In some embodiments, one or more of R₃, R₇, or R₁₂ may include a guanidine group as a cationic functional group and may be bonded to the steroid backbone by an ether linkage. For example, one or more of R₃, R₇, or R₁₂ may be a guanidinoalkyloxy group. An example includes H₂N—C(═NH)—NH-alkyl-O—,

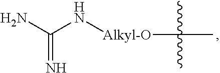

wherein the alkyl portion is defined as with the embodiments described above. In a preferred embodiment, the alkyl portion is a straight chain with 3 carbon atoms, and therefore one or more of R₃, R₇, or R₁₂ may be a guanidinopropyloxy group.

One of skill in the art will recognize that other cationic functional groups may be utilized, and that the cationic functional groups may be bonded to the steroid backbone through a variety of other tethers or linkages. For example, the cationic functional groups may be bonded to the steroid backbone by an ester linkage. For example, one or more of R₃, R₇, or R₁₂ may be an aminoalkylcarboxy or guanidinoalkylcarboxy, such as H₂N-alkyl-C(═O)—O— or H₂N—C(═NH)—NH-alkyl-C(═O)—O—, wherein the alkyl portion is defined as with the embodiments described above. In other embodiments, the cationic functional groups may be bonded to the steroid backbone by an amide linkage. For example, one or more of R₃, R₇, or R₁₂ may be an aminoalkylcarbonylamino (i.e. aminoalkylcarboxamido) or guanidinoalkylcarbonylamino (i.e. guanidinoalkylcarboxamido), such as H₂N-alkyl-C(═O)—NH— or H₂N—C(═NH)—NH-alkyl-C(═O)—NH—, wherein the alkyl portion is defined as with the embodiments described above.

Additionally, one of skill in the art will recognize that the tethers may be of varying lengths. For example, the length between the steroid backbone and the cationic functional group (e.g., amino or guanidino group), may be between 1 and 15 atoms or even more than 15 atoms. In other embodiments, the length may be between 1 and 8 atoms. In a preferred embodiment, the length of the tether is between two and four atoms. In other embodiments, there is no tether, such that the cationic functional group is bonded directly to the steroid backbone.

One of skill in the art will also note that the various cationic functional groups of the present disclosure may be utilized in combination, such that one or more of R₃, R₇, or R₁₂ may include one variation of cationic functional group while one or more of another of R₃, R₇, or R₁₂ of the same compound may include a different variation of cationic functional group. Alternatively, two or more of R₃, R₇, or R₁₂ may include the same cationic functional group, or all of R₃, R₇, or R₁₂ may include the same cationic functional group (in embodiments where all of R₃, R₇, or R₁₂ are cationic functional groups).

Additionally, although in a preferred embodiment one or more cationic functional groups are disposed at R₃, R₇, or R₁₂, one of skill in the art will recognize that in other embodiments, R₃, R₇, or R₁₂ may not be cationic functional groups and/or one or more cationic functional groups may be disposed at other locations of the steroid backbone. For example, one or more cationic functional groups may be disposed at R₁, R₂, R₃, R₄, R₆, R₇, R₁₁, R₁₂, R₁₅, R₁₆, R₁₇, and/or R₁₈.

The compounds and compositions disclosed herein are optionally prepared as salts. The term “salt” as used herein is a broad term, and is to be given its ordinary and customary meaning to a skilled artisan (and is not to be limited to a special or customized meaning), and refers without limitation to a salt of a compound. In some embodiments, the salt is an acid addition salt of the compound. Salts can be obtained by reacting a compound with inorganic acids such as hydrohalic acid (e.g., hydrochloric acid or hydrobromic acid), sulfuric acid, nitric acid, and phosphoric acid. Salts can also be obtained by reacting a compound with an organic acid such as aliphatic or aromatic carboxylic or sulfonic acids, for example formic acid, acetic acid, propionic acid, glycolic acid, pyruvic acid, malonic acid, maleic acid, fumaric acid, trifluoroacetic acid, benzoic acid, cinnamic acid, mandelic acid, succinic acid, lactic acid, malic acid, tartaric acid, citric acid, ascorbic acid, nicotinic acid, methanesulfonic acid, ethanesulfonic acid, p-toluensulfonic acid, salicylic acid, stearic acid, muconic acid, butyric acid, phenylacetic acid, phenylbutyric acid, valproic acid, 1,2-ethanedisulfonic acid, 2-hydroxyethanesulfonic acid, benzenesulfonic acid, 2-naphthalenesulfonic acid, or naphthalenesulfonic acid. Salts can also be obtained by reacting a compound with a base to form a salt such as an ammonium salt, an alkali metal salt, such as a lithium, sodium or a potassium salt, an alkaline earth metal salt, such as a calcium, magnesium or aluminum salt, a salt of organic bases such as dicyclohexylamine, N-methyl-D-glucamine, tris(hydroxymethyl)methylamine, C₁-C₇ alkylamine, cyclohexylamine, dicyclohexylamine, triethanolamine, ethylenediamine, ethanolamine, diethanolamine, triethanolamine, tromethamine, and salts with amino acids such as arginine and lysine; or a salt of an inorganic base, such as aluminum hydroxide, calcium hydroxide, potassium hydroxide, sodium carbonate, sodium hydroxide, or the like.

In some embodiments, the salt is a hydrochloride salt. In some embodiments, the salt is a mono-hydrochloride salt, a di-hydrochloride salt, a tri-hydrochloride salt, or a tetra-hydrochloride salt. Additional examples of salts include sulfuric acid addition salts, sulfonic acid addition salts, disulfonic acid addition salts, 1, 5-naphthalenedisulfonic acid addition salts, sulfate salts, and bisulfate salts.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. An implantable medical device, the implantable medical device comprising:
a structural component formed at least in part from a polymeric material; and
one or more CSA compounds, the one or more CSA compounds being incorporated into the polymeric material of the structural component so as to be distributed throughout the structural component.

2. The medical device of claim 1, wherein the medical device is a catheter, endotracheal tube, intravenous line, feeder tube, drain, prosthesis component, peristaltic pump component, tympsanostomy tube, or tracheostomy tube.

3. The medical device of claim 1, wherein the polymeric material is extrudable.

4. The medical device of claim 1, wherein the polymeric material is a thermoset polymer.

5. The medical device of claim 1, wherein the polymeric material comprises silicone.

6. The medical device of claim 1, further comprising a coating disposed on a surface of the structural component of the medical device, the coating including one or more CSA compounds incorporated therein.

7. The medical device of claim 6, wherein the coating comprises a hydrogel.

8. The medical device of claim 6, wherein the coating is configured as a lubricious coating reducing the coefficient of friction of the surface of the medical device upon which it is disposed by a factor of 5 to 30.

9. The medical device of claim 6, wherein the one or more CSA compounds in the structural component or the coating includes CSA-131 or a salt thereof.

10. The medical device of claim 9, wherein the one or more CSA compounds includes an NDSA salt of CSA-131.

11. The medical device of claim 6, wherein the medical device is an endotracheal tube, and wherein the coating is applied only to a distal tip of the endotracheal tube.

12. The medical device of claim 1, wherein the one or more CSA compounds includes one or more sulfonic acid addition salts.

13. The medical device of claim 12, wherein the one or more sulfonic acid addition salts includes a 1,5-naphthalenedisulfonic acid salt.

14. The medical device of claim 1, wherein the medical device provides protection against biofouling longer than a medical device not having one or more incorporated CSA compounds.

15. The medical device of claim 1, wherein the medical device provides enhanced anti-inflammatory activity as compared to a medical device not having one or more incorporated CSA compounds.

16. The medical device of claim 1, wherein the medical device is an endotracheal tube, and wherein the endotracheal tube better maintains the integrity of the tracheal mucosa when used in intubation as compared to an endotracheal tube not having one or more incorporated CSA compounds.

17. An endotracheal tube, comprising:
a catheter component; and
a coating disposed on at least a portion of a surface of the catheter component, the coating including one or more CSA compounds so as to enable one or more of enhanced antimicrobial activity, enhanced anti-inflammatory activity, and enhanced tissue healing activity, as compared to an endotracheal tube not having a coating with one or more CSA compounds.

18. The endotracheal tube of claim 17, wherein the coating is formed as a hydrogel.

19. The endotracheal tube of claim 17, wherein the one or more CSA compounds includes CSA-131 or a salt thereof.

20. A method of manufacturing an implantable medical device having enhanced antimicrobial, anti-inflammatory, and/or tissue healing properties, the method comprising:
providing a biologically compatible polymeric material;
mixing one or more CSA compounds with the moldable polymeric material; and
forming the moldable polymeric material into an implantable medical device, the one or more CSA compounds thereby being incorporated into the medical device to provide the enhanced antimicrobial, anti-inflammatory, and/or tissue healing properties.

21. The method of claim 20, wherein the polymeric material is formed into the implantable medical device by extrusion.

22. The method of claim 20, wherein the polymeric material includes silicone, and wherein the one or more CSA compounds includes a naphthalenedisulfonic acid (NDSA) salt of a CSA compound.

23. The method of claim 22, wherein the naphthalenedisulfonic acid (NDSA) salt of a CSA compound is the NSDA salt of CSA-131.
